(19) United States
(12) Patent Application Publication
(10) Pub. No.: US 2007/0292459 A1
(43) Pub. Date: Dec. 20, 2007

(54) Halloysite microtubule processes, structures, and compositions

(76) Inventors: Sarah M. Cooper, Mountain View, CA (US); Amara Angelica, Webster, NY (US)

Correspondence Address: BASCH & NICKERSON LLP, 1777 PENFIELD ROAD, PENFIELD, NY, 14526, US

(21) Appl. No.: 11/183,417

(22) Filed: Jul. 18, 2005

(51) Int. Cl. A61K 9/14; A61K 8/02

(52) U.S. Cl. 424/401; 424/489

(57) ABSTRACT

A composition for use in the delivery of a first active agent and a second active agent, the first active agent being sequentially deliverable after the second active agent, the composition comprising hollow mineral microtubules, each of the hollow mineral microtubules having a length, and opposing ends; and including a cylindrical wall, and a hollow lumen; and a central region, an intermediate region, and an outer region along the length; and within the microtubules, the first active agent disposed within the lumen in the central region of the microtubules, and the second active agent disposed within the lumen in the intermediate region of the microtubules. The composition may also be provided for the delivery of three or more active agents in sequence. There is further provided methods for making the compositions of the present invention.

### BACKGROUND OF THE INVENTION

1. Field of the Invention

Compositions and products comprised microtubules of halloysite clay filled with active agents, and methods for preparation thereof.

2. Description of Related Art

In recent years, there have been significant advances in the preparation of new compositions of matter (and uses thereof and/or products made therefrom), such materials including microscopic tubular particles, also known in the art as tubules, microtubules, nanotubules, microtubes, and nanotubes. In certain contexts, such particles may also be referred to as rods or needles or fibers. One example of such tubular particles is the carbon nanotube, which, in various forms, may have a diameter of between about one nanometer and several hundred nanometers, and a length of up to several thousand nanometers long.

These nanotubes, and many other microtubular materials do not occur in nature, or at least not in substantial quantities that make such microtubular materials useful in formulating compositions of matter and/or products in high volume at low cost. Such microtubular materials typically must be synthesized, usually in gram-sized or smaller quantities, resulting in unit manufacturing costs for compositions or products including such microtubular materials that are exceedingly high.

In contrast, there is one type of inorganic microtubular material that does occur in nature in large quantities in mineral form. Such material belong to the kaolinite group of clay minerals, and is described in U.S. Pat. No. 5,651,976, “Controlled release of active agents using inorganic tubules,” of Price et al., the disclosure of which is incorporated herein by reference. In this patent, Price et al. describe the kaolinite group of minerals as follows:

“Several naturally occurring minerals will, under appropriate hydration conditions, form tubules and other microstructures suitable for use in the present invention. The most common of these is halloysite, an inorganic aluminosilicate belonging to the kaolinite group of clay minerals. See generally, Bates et al., ‘Morphology and structure of endellite and halloysite”, American Minerologists 35 463-85 (1950), which remains the definitive paper on halloysite. The mineral has the chemical formula Al₂O₃.2SiO₂.nH₂O. In hydrated form the mineral forms good tubules. In dehydrated form the mineral forms broken, collapsed, split, or partially unrolled tubules.

“The nomenclature for this mineral is not uniform. In the United States, the hydrated tubule form of the mineral is called endellite, and the dehydrated form is called halloysite. In Europe, the hydrated tubule form of the mineral is called halloysite, and the dehydrated form is called is called meta-halloysite. To avoid confusion, mineralogists will frequently refer to the hydrated mineral as halloysite 10 Å and the dehydrated mineral as halloysite 7 Å.

Bates et al. present data on the tubes, which is summarized below:

| | Range (Å) | Median (Å) |
|---|---|---|
| Tube diameter: | 400-1900 | 700 |
| Hole diameter: | 200-1000 | 400 |
| Wall thickness: | 100-700 | 200 |

“Tube lengths range from 0.1 to about 0.75 μm. Morphologically, both hydrated and dehydrated halloysite comprise layers of single silica tetrahedral and alumina octahedral units. They differ in the presence or absence of a layer of water molecules between the silicate and alumina layers. The basal spacing of the dehydrated form is about 7.2 Å and the basal spacing of the hydrated form is about 10.1 Å. (hence the names halloysite 7 Å and halloysite 10 Å). The difference, about 2.9 Å, is about the thickness of a monolayer of water molecules.

“A theory for the formation of hollow tubular microcrystals is presented in Bates et al. Water molecules interposed between the gibbsite (Al₂O₃) and silicate (2SiO₂) layers results in a mismatch between the layers, which is compensated by curvature of the layers.

“Halloysite 10 Å dehydrates to halloysite 7 Å at about 110° C. All structural water is lost at about 575° C. The interlayer water in halloysite 10 Å may be replaced by organic liquids such as ethylene glycol, di- and triethylene glycol, and glycerine.

“Another mineral that will, under appropriate hydration conditions, form tubules and other microstructures is imogolite.

“Another mineral that will, under appropriate conditions, form tubules and other microstructures is cylindrite. Cylindrite belongs to the class of minerals known as sulfosalts.

“Yet another mineral that will, under appropriate conditions, form tubules and other microstructures is boulangerite. Boulangerite also belongs to the class of minerals known as sulfosalts.”

In addition, the term “hydrated halloysite” is used in the claims of U.S. Pat. No. 4,019,934 of Takayama et al., the entire disclosure of which is hereby incorporated by reference into this specification. Claim 1 of this patent refers to an “inorganic gel.” Claim 4 of the patent recites that “4. The inorganic gel-ammonium nitrate composite material as claimed in claim 1 wherein said inorganic gel is prepared from a material selected from the group consisting of hydrated halloysite and montmorillonite.” As is disclosed in column 1 of such patent, “The purified and swollen inorganic gel prepared from a clay such as montmorillonite group, vermiculite, hydrated halloysite, etc., by the manner described hereinafter contains free water, bound water, and water of crystallization. . . . ”

In U.S. Pat. No. 5,651,976 of Price et al., there is disclosed and claimed in claim 1, “A composition for use in the delivery of an active agent at an effective rate for a selected time, comprising: hollow mineral microtubules selected from the group consisting of halloysite, cylindrite, boulangerite, and imogolite, wherein said microtubules have inner diameters ranging from about 200 Å to about 2000 Å, and have lengths ranging from about 0.1 μm to about 2.0 μm, wherein said active agent is selected from the group consisting of pesticides, antibiotics, antihelmetics, antifouling compounds, dyes, enzymes, peptides. bacterial spores, fungi, hormones, and drugs and is contained within the lumen of said microtubules, and wherein outer and end surfaces of said microtubules are essentially free of said adsorbed active agent.”

In claim 11 of U.S. Pat. No. 5,651,976 of Price et al., there is disclosed and claimed, “A composition for use in the delivery of an active agent, at an effective rate for a selected time, into a fluid use environment wherein said active agent has a limited solubility, comprising: hollow cylindrical mineral microtubules selected from the group consisting of halloysite, cylindrite, boulangerite, and imogolite, wherein said microtubules have inner diameters ranging from about 200 Å to about 2000 Å, and have lengths ranging from about 0.1 μm to about 2.0 μm, wherein said active agent is selected from the group consisting of pesticides, antibiotics, antihelmetics, antifouling compounds, dyes, enzymes, peptides, bacterial spores, fungi, hormones, and drugs and is adsorbed onto an inner surface of said microtubules, wherein said microtubules are adherently coated with a coating, wherein said coating is wettable by said fluid and by said active agent, and wherein said coating is permeable to said active agent.”

Further information on the use of halloysite tubules for controlled delivery of active agents is disclosed in U.S. Pat. No. 5,705,191, “Sustained delivery of active compounds from tubules, with rational control,” of Price et al., the disclosure of which is incorporated herein by reference. In this patent, Price et al. disclose a method for releasing an active agent into a use environment, by disposing such active agent within the lumen of a population of tubules, and disposing such tubules into a use environment, either directly or in some matrix such as a paint in contact with the use environment. The tubules have a preselected release profile to provide a preselected release rate curve. The preselected release profile may be achieved by controlling the length or length distribution of the tubules, or by placing degradable endcaps over some or all of the tubules in the population, or by combinations of these methods. Price et al. further disclose a preferred population of tubules having a preselected release profile to provide a preselected release rate curve for controlled delivery of the active agent. In this patent, release rates are expressed in terms of Fick's second law for unsteady state diffusion, and in terms of certain tubule length distributions.

Yet another embodiment for a method involving the use of halloysite tubules is disclosed in U.S. Pat. No. 6,401,816, “Efficient method for subsurface treatments, including squeeze treatments” of Price et al., the disclosure of which is incorporated herein by reference. In this patent, Price et al. disclose a method for delivering encapsulated materials to a subsurface environment, for the treatment of such subsurface environment, having the steps of: (a) loading the lumen of hollow microtubules with an active agent selected for treating the subsurface environment, where the hollow microtubules are compatible with the subsurface environment; and (b) administering the hollow microtubules to the subsurface environment, permitting the controlled release of the active agent into the subsurface environment. The method may be practiced using a slurry of hollow microtubules, where the lumen of these microtubules is loaded with an agent for the treatment of petroleum well environments, and where these loaded microtubules are dispersed in a liquid phase carrier selected from aqueous carriers, non-aqueous carriers, and emulsions of aqueous and non-aqueous materials. The method may also be practiced using a pill made of a consolidated mass of tubules loaded with one or more active agents, typically bound with a binder. This method of Price et al is particularly related to treating subsurface liquid reservoirs, particularly oil reservoirs. More particularly, the method relates to treating oil reservoirs to prevent and/or remedy such problems as fouling of extraction wells by scale formation, well corrosion, and souring of oil by bacterial contamination, and to treating the liquid in such reservoirs by introducing chemical or biological agents, to affect the properties of the liquid or to aid in the extraction of the liquid.

U.S. Pat. No. 4,364,857, “Fibrous clay mixtures,” of Santilli discloses an application not involving the delivery of active agents from halloysite tubules, but rather the use of halloysite tubules in forming a catalyst support and a catalyst composition which have a large pore volume in 200-700 Angstroms diameter pores. With respect to a composition of matter, Santilli discloses, “codispersed rods of a first fibrous clay and a second fibrous clay, the first fibrous clay composed predominantly of fibers with a length range of 0.5-2 microns and a diameter range of 0.04-0.2 microns and a second fibrous clay predominantly composed of rods having a length range of 1-5 microns and a diameter range of 50-100 Angstroms. A preferred first clay is the tubular form of the clay halloysite and a preferred second clay is fibrous attapulgite. It is preferred that the composition be at least 5 percent attapulgite. It is preferred that the composition contain up to 15 percent of a binding refractory inorganic oxide. It is preferred that the refractory inorganic oxide be alumina. It is preferred that the catalyst body have a total pore volume of at least 0.35 cc/g and at least 60 percent of the volume of the pores is present in pores having diameters of 200-700 Angstroms. It is preferred that the composition also include at least one metal selected from the transition metals. This invention also comprises a method for hydroprocessing hydrocarbonaceous feedstocks comprising contacting the feedstocks with molecular hydrogen under hydroprocessing conditions in the presence of a catalyst having codispersed rods of a first fibrous clay having rods predominantly in the range of 0.5-2 microns with a diameter range of 0.04-0.2 microns and a second fibrous clay having rods in the range of 1-5 microns and a diameter range of 50-100 Angstroms. It is preferred that the first fibrous clay be halloysite and the second clay be attapulgite.”

With regard to the halloysite tubules, U.S. Pat. No. 4,364,857 of Santilli further discloses that, “The clay halloysite is readily available from natural deposits. It can also be synthesized, if desired. In its natural state, halloysite often comprises bundles of tubular rods or needles consolidated or bound together in weakly parallel orientation. These rods have a length range of about 0.5-2 microns and a diameter range of about 0.04-0.2 microns. Halloysite rods have a central co-axial hole approximately 100-300 Angstroms in diameter forming a scroll-like structure.

“It has been found that halloysite can make a suitable catalyst for use in demetalizing and hydroprocessing asphaltenes. The halloysite is processed to break up the bundles of rods so that each rod is freely movable with respect to the other rod. When substantially all the rods are freely movable with respect to all the other rods, the rods are defined herein as ‘dispersed’. When the dispersed rod clay is dried and calcined, the random orientation of the rods provides pores of an appropriate size for hydroprocessing and hydrodemetalizing asphaltene fractions.

“When halloysite rods or other rods of similar dimensions are agitated in a fluid such as water to disperse the rods, the dispersion can be shaped, dried and calcined to provide a porous body having a large pore volume present as 200-700 Angstroms diameter pores. When the shaping is by extrusion, however, it has been found that mixtures of dispersed clay rods of the halloysite type, do not extrude well. The rods on the surface of the extruded bodies tend to realign, destroying the desirable pore structure at the surface of the catalyst. This is defined herein as a ‘skin effect’. It has been discovered, however, that if a second fibrous clay with longer, narrower and presumably more flexible, fibers is codispersed with the halloysite-type clay, the resulting composition is easily extrudible, and there is no significant skin effect. ‘Codispersed’ is defined herein as having rod- or tube-like clay particles of at least two distinct types substantially randomly oriented to one another.”

It will be apparent from the disclosures of these United States patents of Price et al., and of Santilli, and from other known art pertaining to the controlled delivery of active agents from microtubules, that in many circumstances, it is desirable to provide and use a population of tubules for which the degree of purity and the tubule diameter and/or length distribution are known, and are preferably deterministically selectable. For the various active agents disclosed by Price et al. in the '976 patent, i.e., “pesticides, antibiotics, antihelmetics, antifouling compounds, dyes, enzymes, peptides, bacterial spores, fungi, hormones, and drugs,” it will be apparent that in processes and comprising such active agents, it will be necessary to deliver such active agents with a high degree of control. Further details on halloysite tubule diameter and/or length distribution may also be found in “Characterization of halloysite for use as a microtubular drug delivery system,” Levis et al., International Journal of Pharmaceutics 243 (2002),

Halloysite is mined and sold commercially from mines in New Zealand and in Juab County, Utah. Reference may be had to http://www.atlasmining.com/dragonmine.html, the web site of the Atlas Mining Company of Osborn, Id. which describes and shows certain operations of the Dragon Mine in the Tintic Mining District in Joab County, Utah. The halloysite clay obtained from the Dragon Mine is among the highest in purity and in proportion of microtubules, and as such is particularly suitable for synthesizing compositions and products in which such tubules are loaded with active agents that are subsequently released in a controlled manner.

There is a need to provide such products for a variety of uses and applications, such as health products, cosmetics, foodstuffs, electronics, engineered polymers, and powders.

Accordingly, embodiments of the present invention are provided herein that meet at least one or more of the following objects of the present invention.

It is an object of this invention to provide a composition for use in the delivery of a first active agent and a second active agent, the first active agent being sequentially deliverable after the second active agent.

It is an object of this invention to provide a method for preparing a composition for use in the delivery of a first active agent, and a second active agent, the first active agent being sequentially deliverable after the second active agent.

It is an object of this invention to provide a cosmetic composition for removal of unwanted hair from skin comprised of halloysite microtubules filled with at least one active agent including a depilatory material.

It is an object of this invention to provide a cosmetic composition for removal of unwanted hair from skin further comprised of halloysite microtubules filled with an additional active agent including a moisturizing material.

It is an object of this invention to provide a cosmetic composition for removal of unwanted hair from skin further comprised of halloysite microtubules filled with an additional active agent including a topical anesthetic material.

It is an additional object of this invention to provide a method for preparing microtubules containing multiple active agents wherein such tubules which have the capability for timed sequential release of such multiple active agents.

### SUMMARY OF THE INVENTION

In accordance with the present invention, there is provided a composition for use in the delivery of a first active agent and a second active agent, said first active agent being sequentially deliverable after said second active agent, said composition comprising hollow mineral microtubules, each of said hollow mineral microtubules having a length, and opposing ends; and including a cylindrical wall, and a hollow lumen; and a central region, an intermediate region, and an outer region along said length; and within said microtubules, said first active agent disposed within said lumen in said central region of said microtubules, and said second active agent disposed within said lumen in said intermediate region of said microtubules.

In accordance with the present invention, there is further provided a composition for use in the delivery of a first active agent, a second active agent, and a third active agent, said first active agent being sequentially deliverable after said second active agent, and said second active agent being sequentially deliverable after said third active agent, said composition comprising hollow mineral microtubules, each of said hollow mineral microtubules having a length, and opposing ends; and including a cylindrical wall, and a hollow lumen; and a central region, an intermediate region, and an outer region along said length; and within said microtubules, said first active agent disposed within said lumen in said central region of said microtubules, said second active agent disposed within said lumen in said intermediate region of said microtubules, and said third active agent disposed within said lumen in said outer region of said microtubules.

In accordance with the present invention, there is further provided a method for preparing a composition for use in the delivery of a first active agent, and a second active agent, said first active agent being sequentially deliverable after said second active agent, said method comprising the steps of providing hollow mineral microtubules, each of said hollow mineral microtubules having a length, and opposing ends; and including a cylindrical wall, and a hollow lumen; and a central region, an intermediate region, and an outer region along said length; filling said hollow mineral microtubules with said first active agent; eluting said first active agent in said microtubules, thereby removing said first active agent from said outer region and said intermediate region of said microtubules; and filling said intermediate region with said second active agent.

In accordance with the present invention, there is further provided a method for preparing a composition for use in the delivery of a first active agent, a second active agent, and a third active agent, said first active agent being sequentially deliverable after said second active agent, and said second active agent being sequentially deliverable after said third active agent, said method comprising the steps of providing hollow mineral microtubules, each of said hollow mineral microtubules having a length, and opposing ends; and including a cylindrical wall, and a hollow lumen; and a central region, an intermediate region, and an outer region along said length; filling said hollow mineral microtubules with said first active agent; eluting said first active agent in said microtubules, thereby removing said first active agent from said outer region and said intermediate region of said microtubules; filling said intermediate region and said outer region of said microtubules with said second active agent; eluting said second active agent in said microtubules, thereby removing said second active agent from said outer region of said microtubules; and filling said outer region with said third active agent.

In the compositions and methods of the present invention, the hollow mineral microtubules may be halloysite, cylindrite, boulangerite, and imogolite microtubules, and mixtures thereof. The microtubules are preferably halloysite microtubules.

The microtubules may be further provided with degradable endcaps, such that the active agents are contained therein until they are applied to the desired location for active agent delivery.

In various embodiments, the active agents may be substances that provide a hair removal composition, wherein the first agent is a depilatory agent, the second agent is a moisturizing agent, and the third agent is an anesthetic agent. In one embodiment, the moisturizing agent is not present.

The filled tubules may be dispersed in a carrier fluid for fluid transport to the desired location for active agent delivery.

### BRIEF DESCRIPTION OF THE DRAWINGS

The invention will be described by reference to the following drawings, in which like numerals refer to like elements, and in which:

FIG. 1 is an end view of a halloysite nanotubule containing active agents for the purpose of effecting the removal of unwanted hair from skin;

FIG. 2 is a cross-sectional view of the filled tubule of FIG. 1, taken along the lines 2-2 of FIG. 1;

FIGS. 3A-3F are cross-sectional sequential views of a halloysite nanotubule as it undergoes the process of being filled with several active agents;

FIG. 4A is a cross-sectional view of a one halloysite tubule that may be incorporated into a lipstick formulation wherein the active agent in the tubule includes a colorant;

FIG. 4B is a cross-sectional view of a one halloysite tubule that may be incorporated into a lipstick formulation wherein the active agent in the tubule includes a skin moisturizer; and

FIGS. 5A and 5B are schematic representations of the use of a lipstick formulation containing halloysite tubules loaded with an active agent.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

For a general understanding of the present invention, reference is made to the drawings. In the drawings, like reference numerals have been used throughout to designate identical elements. In describing the present invention, a variety of terms are used in the description. Standard terminology is widely used in mineral processing, separation, and purification art. For example, one may refer to the SME Mining Engineering Handbook, 2nd Ed. Volume 1, 1992, published by the Society for Mining, Metallurgy, and Exploration. One may also refer to Mineral Processing Technology, 6^{th} Ed. B. A. Wills, Butterworth Heinemann, 1997.

For general reference in regard to the ingredients and manufacture of cosmetics, reference may be had to “A Consumer's Dictionary of Cosmetic Ingredients: Complete Information About the Harmful and Desirable Ingredients in Cosmetics and Cosmeceuticals,” R. Winter, 6^{th} Edition, Three Rivers Press, 2005; hereinafter abbreviated “CDCl of R. Winter,” or the “CDCl.”

For general reference in regard to the ingredients and manufacture of cosmetics, reference may be had to “The Beauty Bible,” S. Stacey et al., The Overlook Press, Peter Mayer Publishers, Inc., 1997 hereinafter abbreviated, “Beauty Bible of S. Stacy,” or “Beauty Bible.”

With regard to the handling of multiphase halloysite-containing fluids, a variety of terms are used in the description. As used herein, a two phase fluid is meant to be a fluid comprising a liquid phase in which either substantially solid particles are dispersed therethrough, or a first liquid phase in which droplets or particles of a second liquid phase immiscible with such first liquid phase are dispersed through such first liquid phase. A multiphase fluid is meant to be a fluid comprising a first liquid phase in which at least one additional second solid or liquid phase is dispersed therethrough.

As used herein, a “particle” is meant to be a discreet liquid droplet or a solid object, with a characteristic dimension such as a diameter or length of between about one nanometer, and about several centimeters. The particular size of particles in a fluid being processed will depend upon the particular application.

As used herein, a “dispersion” is meant to be any fluid comprising a liquid phase in which substantially solid particles are suspended, and remain suspended, at least temporarily.

As used herein, a “slurry” is meant to be any fluid comprising a liquid phase in which substantially solid particles are present. Such particles may or may not be suspended in such fluid.

As used herein, the terms “microtubule” and “nanotubule” are used interchangeably, and are to be taken to mean a microscopic cylindrical tubular shaped material entity having a diameter between about 10 and about 500 nanometers, and a length of between about 100 and about 5000 nanometers.

As used herein, the terms “cosmetic” and “cosmetics” are meant to indicate materials that are used to affect the appearance of, or improve the health of human skin, hair, nails, and other body surfaces.

A variety of super absorbent compositions and products that comprise nanotubules containing an active agent may be made, including but not limited to fiber-based diapers and feminine personal care products, chemical spill absorbers, and odor absorbents.

A variety of health care compositions and products that comprise nanotubules containing an active agent may be made, including but not limited to anti-bacterial fiber/paper products such as diapers, sponges, paper towels, tissues, baby wipes, paper plates, napkins, and pantry coverings; anti-fungal/anti-mold coatings, paints, and sealants; restorable bone materials; pharma mini-reactors for drug profiling and/or drug interaction testing; therapeutic contrast agents; wound-healing bandages and dressings; systemic drug delivery; targeted drug delivery; dental implants; and low-toxicity tick and flea treatments.

A variety of cosmetic compositions and products that comprise nanotubules containing an active agent may be made, including but not limited to facial cosmetics such as longer lasting lipsticks, eyeliners, color enhancers, light reflectors, volumizers, and mascara; lotions and creams such as replenishing moisturizer, replenishing sunscreen, skin balancers, acne cleaners, foaming cleansers, face rubs, anti-wrinkle creams, smoothing lotions, anti-cellulite lotions; longer lasting perfumes; collagen treatments as alternatives to elective surgery; anti-itch creams, and hair and nail products such as hair conditioning treatments, hair removal treatments, hair growth prevention treatments, hair growth promotion treatments, and anti-crack nail-polishes.

One particular useful formulation containing filled halloysite nanotubules is a hair removal formulation. The use of such a formulation obviates the need for other methods of hair removal, such as shaving, electrolysis, tweezing, waxing, and direct chemical treatments, each of which has certain disadvantages. Shaving, for instance, removes only the exposed surface of the hair, requiring repeated treatments every 2-3 days, and after continued use, may cause hair to grow back thicker and fuller than prior to shaving. Furthermore, the risk of nicks and cuts negatively affects the desired smooth, clean appearance and feel. Electrolysis, the process of permanently removing hair by applying an electric current to the hair follicle below the skin, takes many repeated trips to a professional which is expensive and time consuming, and carries the risk of electric shock, scarring, and inconsistent results. Further details on the issues with this technique may be found at page 297 in the “Beauty Bible” of S. Stacy.

Tweezing, pulling a hair out by the root, is generally used only for the facial area as it is painful, time consuming, and can also cause scars. Waxing, like tweezing, yanks the hair from the follicle and generally lasts 5-7 days. As noted previously herein, waxing is a painful process. Hot waxing can cause burns, scarring, and skin irritation. Depilatory creams or chemical hair removal treatments employ strong pH ingredients to dissolve the hair. According to the aforementioned “Beauty Bible” of S. Stacy, at page 292 therein, “Hair and skin are similar in composition, so chemicals that destroy the hair can also destroy the skin.” This results in risks of chemical burns, severe skin irritation, and allergic responses to active ingredients.

The advantages of halloysite use in depilatories and other hair removal schemes are that by loading the tubules with potentially damaging active ingredients, collateral damage to surrounding skin can be minimized. It has been observed that the pigmentation and other ingredients in make-up creams such as foundation end up collecting in pores and fine lines in the skin. “Most foundations contain ingredients that allow some amount of movement . . . that also means that those foundations can easily slip into pores . . . Moisturizing when you don't need it creates even more slippage . . . Too much moisturizer or too much foundation can absolutely cause slippage into lines and pores.” (“Beauty Bible,” pp 501-502.) Hair follicles are found in those same pores. This migration or slippage is due to the use of emollients in those creams. By turning this disadvantage of make-up creams to an attribute, and using it to deliver the filled tubes directly to the hair follicle, using an emollient based carrier cream or lotion, the tubes will release their contents specifically to the desired follicle. Furthermore, this approach also allows for more effective use of these harmful chemicals and thereby allows lower levels of the potential irritant or allergen to be used.

There may be other ingredients loaded into the tubes where the desire is not to reduce harm to surrounding skin, but rather to increase effectiveness of active or functional ingredients such as local anesthetics or antiseptics in waxing, moisturizer or skin refreshener, perfume, and/or skin repair ingredients. This list is meant as a partial list of potential fillers; as is obvious to those skilled in the ordinary art, there are other potential ingredients that may improve the effectiveness or experience or hair removal.

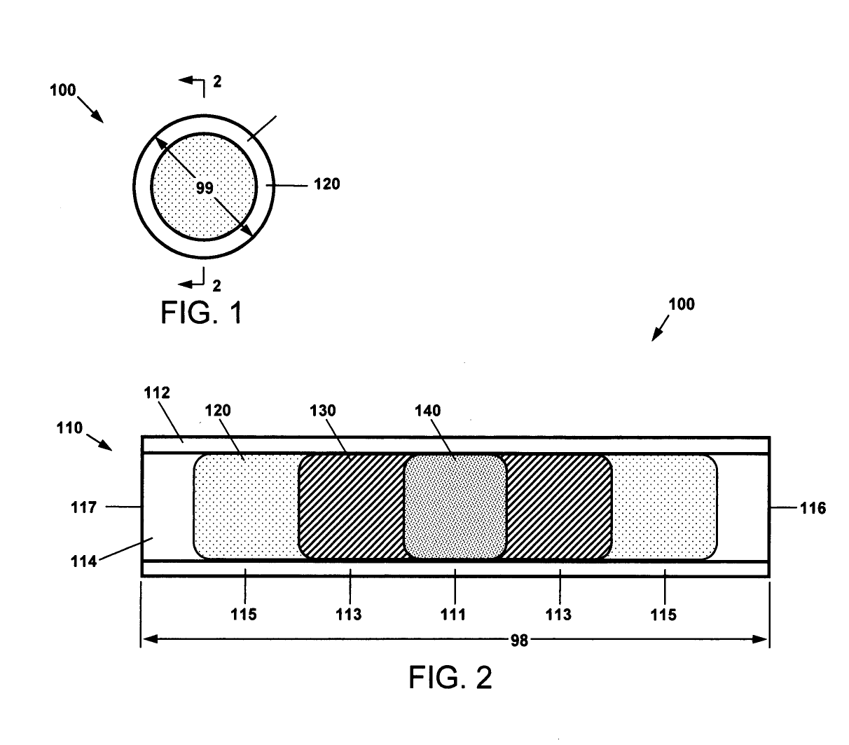

FIG. 1 is an end view of a halloysite nanotubule containing active agents for the purpose of effecting the removal of unwanted hair from skin. FIG. 2 is a cross-sectional view of the filled tubule of FIG. 1, taken along the lines 2-2 of FIG. 1. It is to be understood that for the sake of simplicity of illustration, the halloysite nanotubules depicted herein are rendered as simple thin-walled cylinders, with it being understood that such halloysite nanotubules may have considerably greater wall thickness relative to diameter, and such nanotubules may have a scroll-like rolled up wall shape as disclosed in the aforementioned paper of Bates et al., ‘Morphology and structure of endellite and halloysite”, American Minerologists 35 463-85 (1950).

Referring to FIGS. 1 and 2, filled nanotubule 100 is a generally cylindrical tubule having a diameter 99 and a length 98 between opposite ends 116 and 117. Filled nanotubule 100 comprises an inorganic tubule 110 that preferably consists essentially of halloysite clay. Inorganic tubule 110 is comprised of a substantially cylindrical wall 112 that encloses a hollow lumen 114 disposed between open ends 117 and 116. Inorganic tubule 110 contains active agents for the purpose of effecting the removal of unwanted hair from skin. In one embodiment, hollow lumen 114 contains a hair dissolver material 140 loaded into the central region 111 of tubule 110, a skin moisturizing material 130 (or a skin freshener or astringent) loaded in an intermediate regions 113 of tubule 110, and a topical anesthetic 120 loaded into the outer regions 115 of tubule 110. It is also within the scope of the present invention that one or a combination of these active agents may be present within the tube, while remaining components may be included in the either the tubule or in an accompanying carrier fluid.

To formulate a hair removal formulation, loaded nanotubules are mixed into a carrier fluid. Such carrier fluid is applied to the skin from which is growing unwanted hair. In use, topical anesthetic material 120 first diffuses out of nanotubules 110 and reduces the sensitivity of the skin to irritation. Moisturizer material 130 then diffuses out of nanotubules 110 and softens the skin and/or unwanted hair. Chemical hair dissolver material 140 then diffuses out of nanotubules 110 and dissolves the unwanted hair, either completely, or locally at the follicle. Subsequently, the entire formulation including the dissolved hair remnants may be wiped or washed away, leaving the skin in a moisturized and hairless condition.

In one embodiment, hair dissolver material 140 may consist of active ingredients and inactive or carrier ingredients. Suitable hair dissolver materials, also known as depilatories, include but are not limited to calcium thioglycolate and other thioglycolic acid salts, as well as various sulfides, such as hydrogen sulfide. Reference may be had to the “CDCl” of R. Winter. At page 193 of such book, there is disclosed, “The most effective chemical hair removers yet discovered are the sulfides, particularly hydrogen sulfide, but they have an unpleasant odor that is hard to mask. Most sulfides have been replaced with salts of thioglycolic acid, which take more time to act but smell better and are not as irritating as sulfides.” Additionally, it is noted that, “Cream depilatories that act by dissolving the hair usually contain calcium thioglycolate, calcium carbonate, calcium hydroxide, cetyl alcohol, sodium lauryl sulfate (a detergent), water, and a strong perfume (so as to remain stable in an alkali medium).” In the current embodiment the halloysite tubes may contain one or more of these ingredients.

And at page 151 of the “CDCl,” it is noted that thioglycolic acid compounds are “ . . . prepared by the action of sodium sulfohydrate on sodium chloroacetate. A liquid with a strong unpleasant odor; mixes with water and alcohol. The calcium salts are used in hair depilatories, hair waxing solutions, and lotions. Thioglycolates can cause hair breakage, skin irritations, severe allergic reactions, and pustular reactions . . . . Canadians limit it to 5 percent in depilatories.”

In one embodiment, moisturizer material 130 may consist of active ingredients and an inactive or carrier ingredients. Suitable moisturizer materials include but are not limited to thioglycerol, petrolatum, and/or emollients. Thioglycerol is “Used in soothing skin lotions. Prepared by heating glycerin and alcohol. Yellowish, very viscous liquid, with a slight sulfur odor. Used to promote wound healing.” (“CDCl,” page 515)

Petrolatum, also known as Vaseline, petroleum jelly, and paraffin jelly, “is a purified mixture of semisolid hydrocarbons from petroleum. Yellowish to light amber or white, semisolid unctuous mass, practically odorless and tasteless, almost insoluble in water . . . . it makes creams smoother. Helps to soften and smooth the skin in the same way as any other emollient, and is less expensive. The oily film helps prevent evaporation of moisture from the skin and protects the skin from irritation.” (“CDCl,” page 397)

In one embodiment, topical anesthetic 130 may consist of active ingredients and an inactive or carrier ingredients. Suitable anesthetic materials include but are not limited to Benzocaine, witch hazel, and Procaine. Benzocaine, also known as ethyl aminobenzoate is “A white, crystalline powder slightly soluble in water and a local anesthetic . . . . As an anesthetic, it is reported low in toxicity.” It also used in eyebrow plucking creams and after shave. (“CDCl,” page 102.) Witch Hazel “is a skin freshener, local anesthetic, and astringent made from the leaves and/or twigs of Hamamelis virginiana. Collected in the autumn. Witch hazel has an ethanol content of 70 to 80 percent and a tannin content of 2 to 9 percent.” (“CDCl,” page 546.) Procaine is also known as “Novocain. A local anesthetic.” (“CDCl,” page 426.)

In one embodiment, skin freshener or astringent 130 may consist of active ingredients and an inactive or carrier ingredient(s). As is disclosed at page 461 of the aforementioned “CDCl” of R. Winter, “Fresheners are weaker than astringents. They are usually clear liquids designed to make the skin feel cool, tight, and refreshed.” A suitable freshener formula includes but is not limited to “about 60 percent witch hazel, about 15 percent camphorated alcohol, 24 percent alcohol, and 1 percent citric acid. May also contain arnica, bay rum, boric acid, chamomile, floral scents, glycerin, lactic acid, magnesia, menthol, lavender oil, phosphoric acid, talc, benzoin, and aluminum salts.” And at page 90 of such book, it is noted that an astringent is “a clear liquid containing mostly alcohol, but with small amounts of other ingredients such as boric acid, alum, menthol, and/or camphor. A typical astringent formula is as follows: ethanol, 50 percent; sorbitol, 2.5 percent; perfume oil, 0.1 percent; menthol, 0.1 percent; boric acid, 2.0 percent; water 44.9 percent. In addition to making the skin feel refreshed, it usually gives a tightened feeling from the evaporation of ingredients.”

In one embodiment, skin healing ingredient 130 may consist of active ingredients and one or more of an inactive or carrier ingredient. Suitable healing materials include but are not limited to allantoin, zinc oxide, and propylparaben.

Allantoin is used “to help heal wounds and skin ulcers and to stimulate the growth of healthy tissue. Colorless crystals, soluble in hot water, it is prepared synthetically by the oxidation of uric acid or by heating uric acid with dichloroacetic acid.” (“CDCl,” page 58.)_Zinc Oxide or Flowers of Zinc is “A creamy white ointment used medicinally as an astringent, antiseptic, and protective in skin diseases. Zinc is believed to encourage healing of skin disorders.” (“CDCl,” page 552.) Propylparaben or “Propyl p-Hydroxybenzoate. Developed in Europe, the esters of p-hydroxybenzoic acid are widely used in the cosmetic industry as preservatives and bacteria- and fungus-killers. They are active against a variety of organisms, are neutral, low in toxicity, slightly soluble, and active in all solutions, alkaline, neutral, or acid . . . . Used medicinally to treat fungus infections . . . Less toxic than benzoic or salicylic acid.” (“CDCl,” page 429.)

In one embodiment, hair growth inhibitor ingredient 130 may consist of active ingredients and an inactive or carrier ingredients. Suitable inhibitor ingredients include but are not limited to eflornithine hydrochloride. Manufactured by Bristol-Meyers Squibb as Vaniqa, it “is FDA-approved as a prescription-only topical cream for reducing and inhibiting the growth of unwanted facial hair” (“Beauty Bible,” page 300).

With various suitable materials for hair dissolver 140, moisturizer 130, and topical anesthetic having been identified, an overall method for the preparation of nanotubules 110 filled with the preferred combination of such materials will now be described. It is to be understood that although the following method will be described for the preparation of a hair removal formulation containing halloysite tubules with several active agents, such method described herein is generally applicable to the preparation of a wide range of halloysite tubules containing a plurality of active ingredients, for which a sequential release is desired. Accordingly, the following description of such method for preparation of such hair removal formulation is not to be construed as limited to hair removal formulations.

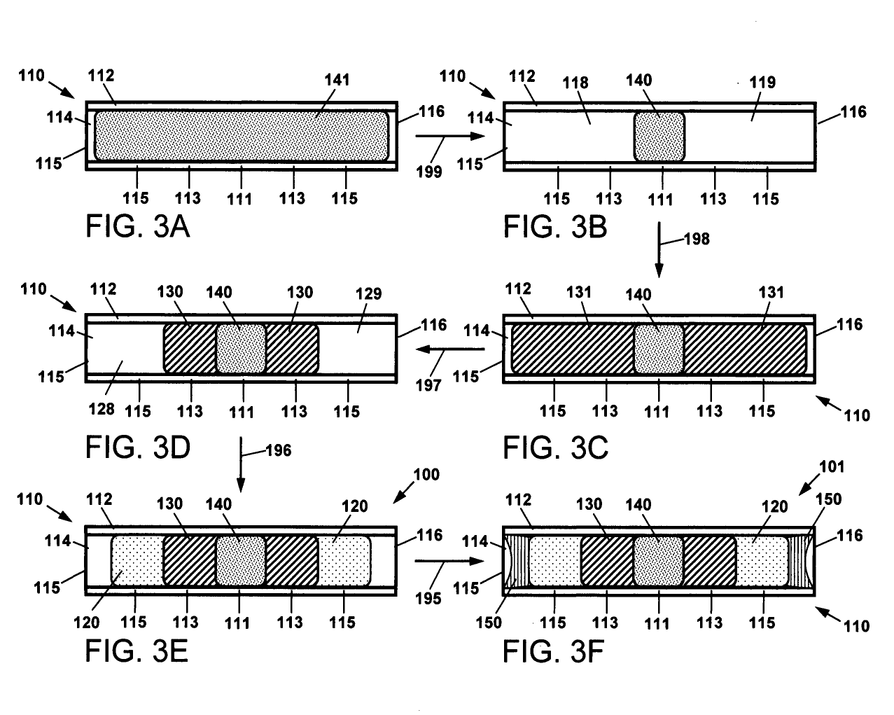

FIGS. 3A-3F are cross-sectional sequential views of a halloysite nanotubule as it undergoes the process of being filled with several active agents. As just indicated previously herein, FIGS. 3A-3F are drawn with reference in particular to the preparation of the multi-agent halloysite tubules previously disclosed herein with reference to FIGS. 1 and 2. Referring to FIG. 3A, in a first step, a batch of inorganic tubules including inorganic tubule 110, which is preferably a halloysite tubule, is first filled with depilatory material 141. Such tubule is filled according to the methods disclosed in U.S. Pat. No. 5,651,976, “Controlled release of active agents using inorganic tubules,” of Price et al., at columns 7-8, under the heading, “Method of entrapment.” The disclosure this United States patent is incorporated herein by reference. (It is further noted that such methods of Price et al. may be used in the loading of tubules with all of the active agents described herein.)

Subsequent to filling, the tubes 110 are placed in slurry with solvent (e.g., water or ethanol for hydrophilic components; or isopropyl alcohol or mineral oil for hydrophobic components) in a step indicated by arrow 199 between FIG. 3A and FIG. 3B such that the depilatory mixture 141 begins to elute out both ends. The solvent/halotube mixture may be heated or actively agitated in order to increase the elution rate (via diffusion). As elution first occurs at the ends of the tube, a void at each tube end will be created. The size of this void will depend on the reaction time within the slurry, the diameter of the tubes used, and the degree to which the depilatory is soluble in the solvent.

After a suitable reaction time where from about 30 percent to about 80 percent of the material 141 has eluted out of lumen 114, the solvent is drained from the slurry and the halotubes are dried to remove excess solvent from the voids, or in the case of mineral oil, cleaned with a mild acid and then dried. Referring to FIG. 3B, the resulting product is a powder or cake containing the partially filled tubules such as tubule 110 containing an amount of depilatory 140 within the lumen 114 at the central region 111 of such tubule.

At this point in the process, a second material such as a moisturizer or local anesthetic can be added by the same method as the depilatory, except this second material will fill into the voids 118 and 119 created at the ends 115 and 116 of the tube 110. It will be apparent that this procedure can be repeated many times to create a tube filled sequentially with several components. Referring to FIGS. 3B and 3C in one preferred embodiment, in step indicated by arrow 198, the tubules such as tubule 110 are then filled with a moisturizing material 131. Subsequently, the tubules are then put through another elution step indicated by arrow 197 in which from about 30 percent to about 80 percent of the material 131 has eluted out of lumen 114, to result in tubules such as tubule 110 of FIG. 3D, that contain depilatory 140 at central region 111, and moisturizing material 130 at intermediate region 113 of tubule 110.

In a subsequent step indicated by arrow 196, the remaining voids 118 and 119 at the ends 115 and 116 of the tubules such as tubule 110 are then filled with a moisturizing material 120 at outer region 115 of tubule 110, resulting the multi-agent filled tubule 100 of FIG. 3E and of FIGS. 1 and 2.

This method of filling tubules with multiple active agents enables the provision of filled tubules which have the capability for timed sequential release of such multiple active agents. In a further embodiment, such filling method may also be used to “cap” the ends of the halotubes such that materials 120, 130, and 140 etc. within lumen 114 cannot be released until a stimulus such as heat or chemical catalyst is present.

For instance, if halotubes were intended to be used in the present application of hair removal to deliver only the local anesthetic component in a waxing type procedure, the halotubes could be included in a pre-wax anesthetic vanishing cream that serves to numb the area before application and subsequent removal of the hot wax. As the tubes allow a higher concentration of the anesthetic to be delivered directly to the skin to maximize the effectiveness of the anesthetic, while allowing minimum use of these chemicals and therefore minimal side effects from their use, pre-elution of the anesthetic into the carrier cream or onto the skin would negate the benefit of using halotubes.

Referring again to FIG. 3E, and referring to FIG. 3F, in order to seal the anesthetic into the tubes 110 until desired release (such as when the hot wax is applied to the hair removal area of the skin), the above described procedure for sequential loading of the tubes is employed in a step indicated by arrow 195 to provide a capping material 150 at tube ends 115 and 116 that would be removed by application of the hot wax (not shown), either through heating and a resulting phase or viscosity change of the capping material 150, or by chemical reaction between the halotube 110 and an ingredient in the wax.

Further details on a process for the provision of endcaps on halloysite tubules is provided in U.S. Pat. No. 5,705,191, “Sustained delivery of active compounds from tubules, with rational control,” of Price et al., at columns 5-6, under the heading, “Providing Tubules with Degradable Endcaps.” The disclosure this United States patent is incorporated herein by reference.

It will be apparent that the relative proportions of the respective materials 140, 130, and 120 may vary, as well as the depth of the endcaps 150 of capped tubule 101 of FIG. 3F. It will be further apparent that the relative shapes of materials 140, 130, and 120 within the lumen 114 of tubules 110 are drawing simply for illustrative purposes in FIGS. 3A-3F. There is no intent to imply that the boundaries of such materials 140, 130, and 120 are always convex in shape as depicted in FIGS. 3A-3F; such boundaries may be concave, as is depicted for capping material 150 in FIG. 3F. It will be further apparent that materials 140, 130, and 120 are not necessarily pure substances, such as a pure depilatory material; but rather such materials may include a carrier material as a diluent, or a dispersant, or having some other function. The disclosure of some of such carrier materials is provided in a section of column 6, titled “E. Carriers” in the aforementioned U.S. Pat. No. 5,651,976, “Controlled release of active agents using inorganic tubules,” of Price et al.

Referring again to capped tubule 101 of FIG. 3F, suitable capping materials 150 that may elute in response to heat include petrolatum or petroleum jelly, which has a high viscosity at room temperature, but becomes runnier (i.e. lower in viscosity) when heated above 35° C. In the case of petrolatum, the caps would be added at 35° C. or higher temperature to shorten diffusion times into the void tube ends, and subsequently washed with a solvent such as ethanol to remove excess petrolatum. After a brief wash, the filled tubes are cooled to cause the petrolatum to reform a jelly state thereby encapsulating the anesthetic 120 contained in region 115 of the tube 110.

Suitable capping agents that respond to pH include Poloxamer 188 (“CDCl” Page 410) “Poloxane. A liquid, nonionic, surfactant polymer. If chain lengths of polyoxyethylene and polyoxypropylene are increased, the product changes from liquid to paste to solid.”

Further suitable materials and properties of degradable endcaps are disclosed in the aforementioned U.S. Pat. No. 5,705,191, “Sustained delivery of active compounds from tubules, with rational control,” of Price et al., at columns 5-6, under the heading, “Providing Tubules with Degradable Endcaps.”

To summarize, in the steps performed through the step 198 resulting in the filled tubule 110 of FIG. 3C, there is provided tubules which are capable of sequentially releasing a second active agent 131, followed by a first active agent 140. In the steps performed through the step 196 resulting in the filled tubule 110 of FIG. 3E, there is provided tubules which are capable of sequentially releasing a third active agent 120, followed by a second active agent 130, followed by a first active agent 140.

Following the preparation of the filled tubules 110 of FIG. 2, an overall hair removal formulation is prepared in which the filled tubules 110 are dispersed in a carrier lotion fluid. In one embodiment, a suitable carrier lotion is a moisturizing lotion containing cetyl alcohol, 0.5 percent; lanolin, 1 percent; stearic acid, 3 percent; glycerin, 2 percent; methylparaben, 0.1 percent; triethanoamine, 0.75 percent; water, 85 percent; and perfume in sufficient amounts, as is disclosed at page 275 of the aforementioned “CDCl” of R. Winter.” It will be apparent that other similar formulations may be used, which contain these ingredients in somewhat varying proportions.

To increase the slippage into pores, the carrier lotion may consist of ingredients traditionally found in foundations but with increased amounts of slippage agents such as glycerin, silicone, and mineral oil. One suitable foundation formula for such use is disclosed at page 250 of the aforementioned “CDCl,” and may be comprised of about 50 percent water, mineral oil, stearic acid, lanolin, cetyl alcohol, propylene glycol, triethanolamine, borax, and insoluble pigments. The formula may also contain emulsifiers and detergents; humectants such as propylene glycol, glycerin, and sorbitol to absorb and retain water; lanolin derivatives; perfume; preservatives such as paraben; special barrier ingredients such as zinc stearate; cellulose derivatives and silicone; synthetic esters; thickeners such as sodium alginate, gum tragacanth, quince seed, and mucilage; and such waxes as beeswax and spermaceti. In one embodiment, the foundation formula is provided without insoluble pigments, and/or without the waxes, as such ingredients may serve to slow the migration of the carrier lotion to pores.

In use, the hair removal formulation is applied topically to the skin. In embodiments requiring a “wax treatment”, the wax-like products, also called epilatories, may be softened by heat and applied when cool and then ‘yanked’ off, from the skin suddenly, taking embedded hair with them. The wax acts by hardening around the hair and pulling it out of the follicle. Such products usually contain low melting temperature waxes such as rosin, beeswax, paraffin, and petrolatum. It is believed that waxing leaves the area smoother than shaving does because it pulls the hair out below the top layer of skin, which makes such hair grow back slower and less uniformly.

The method of hot waxing and “sugaring” are described as follows: “In hot waxing, a thin layer of heated wax is applied to the skin in the direction of hair growth. The hair becomes embedded in the wax as it cools and hardens. The wax is then pulled off quickly in the opposite direction of hair growth, taking the uprooted hair with it. Cold waxes work similarly. Strips precoated with wax, or a cool sugar-based substance are pressed onto the skin in the direction of the hair growth and pulled off in the opposite direction . . . ‘sugaring’ method of hair removal . . . uses sugar instead of wax. With its thick, caramel-like consistency, it works identically to regular waxing . . . Sugaring's mess washes away while wax has to be peeled . . . Plus sugaring doesn't require heating while waxing often does, and adding heat is far more damaging to the skin”

In applications where longer periods between treatments is desired, waxing or ‘sugaring’ treatments provide the best method of hair removal. In another embodiment of the present invention, halloysite tubules can provide initial hair softening by loading such tubules with lower concentrations of the chemical depilatories described previously or with moisturizers, or deliver local anesthetics or astringents to numb the skin prior to hair removal.

In addition to hair removal, halloysite tubules may used to deliver hair growth remedies to pores and subsequently hair follicles directly, increasing the effectiveness of lower levels of active ingredients and providing a longer elution time to increase the actual hair growth experienced. Ingredients that could be loaded into tubules that promote hair growth include but are not limited to minoxidil, tretinoin, retin A, and renova. Ingredients that may stimulate the skin, include but are not limited to rubefacients, such as pilocarpine, that help stimulate blood circulation to the scalp and the activity of oil-secreting glands.

Following the preparation of the filled tubules 110 of FIG. 2, an overall hair growth formulation is prepared in which the filled tubules 110 are dispersed in a carrier lotion fluid such as a moisturizer. In one embodiment, a suitable carrier lotion is a hair thickener lotion that contain oils and proteins that coat the hair with an invisible film, thus giving it “body.” Such thickeners make the hair feel smoother to the touch and more “manageable,” i.e. resistant to tangling and to the effects of static electricity. In one embodiment, a suitable hair thickener lotion may contain resorcinol, 0.8 percent; chloral hydrate, 1.5 percent; ethanol, 80 percent; beta-napthol, 0.8 percent; turkey-red oil, 16.9 percent; perfume; and a colorant, as is disclosed at page 274 of the aforementioned “CDCl” of R. Winter.” It will be apparent that other similar formulations may be used, which contain these ingredients in somewhat varying proportions. Such lotion may also contain one or more of the following ingredients: benzalkonium chloride, camphor, cantharides tincture, phosphoric acid, pilocarpine, glycols, quinine, sorbitan derivatives, salicyclic acid, and tars.

To increase the slippage into pores, the carrier lotion may further consist of ingredients traditionally found in foundations but with increased amounts of slippage agents such as glycerin, silicone, and mineral oil. One suitable foundation formulation to increase slippage is the formulation described previously herein in this specification with regard to hair removal formulations.

Among the cosmetic compositions and products that may comprise nanotubules containing an active agent are facial cosmetics such as longer lasting lipsticks. As is disclosed in the aforementioned “CDCl” of R. Winter, lipstick is described therein as, “Primarily a mixture of oil and wax in a stick form with a red-staining certified dye dispersed in oil, red pigments similarly dispersed, flavoring, and perfume. Bromo acid, D & C Red No. 21, and related dyes are most often used. Among other common lipstick dyes are D & C Red No. 27 and insoluble dyes known as lakes, such as D & C Red, No. 34 Calcium Lake, and D & C Orange No. 17 Lake. Pinks are made by mixing titanium oxide with various reds. Among oils and fats used are olive, mineral, sesame, castor, butyl stearate, polyethylene glycol, cocoa butter, lanolin, petrolatum, lecithin, hydrogenated vegetable oils, carnauba and candelilla waxes, beeswax, ozokerite, and paraffin.”

As is further disclosed in a publication of R. Johnson, “Lipstick” Chemical and Engineering News 77 (28) Pg. 31 Nov. 12, 99), “Lipstick contains a variety of waxes, oils, pigments, and emollients. The wax gives lipstick its shape and ease of application. Among the waxes are beeswax, a substance obtained from bee honeycombs that consists of esters of straight-chain monohydric alcohols with even-numbered carbon chains from C₂₄ to C₃₆ and straight-chain acids also having even numbers of carbon atoms up to C₃₆. Other waxes include carnauba wax, which is an exudate from the pores of leaves of Brazilian wax palm trees, and candelilla wax, which is obtained from the candelilla plant and is produced in Mexico by immersing the plants in boiling water containing sulfuric acid and skimming off the wax that rises to the surface.

“The oils and fats used in lipstick include olive oil, mineral oil, castor oil, cocoa butter, lanolin, and petrolatum. More than 50% of lipsticks manufactured in the U.S. contain substantial amounts of castor oil. It forms a tough, shiny film when it dries after application.

“In recent years, ingredients such as moisturizers, vitamin E, aloe vera, collagen, amino acids, and sunscreen have been added to lipstick. The extra components keep lips soft, moist, and protected from the elements.

“Lipstick gets its color from a variety of added pigments. Among them are bromo acid, D&C Red No. 21, and related dyes. Other common lipstick dyes are D&C Red No. 27 and insoluble dyes known as lakes, such as D&C Red No. 34, Calcium lake, and D&C Orange No. 17.

“From the oven to the store comes a variety of lipsticks: frosted, mattes, sheers, stains, and long-lasting color. Frosted lipsticks include a pearlizing agent—often a bismuth compound—that adds luster to the color. Bismuth oxychloride, which is synthetic pearl, imparts a frost or shine. Bismuth subcarbonate is used as a skin protective. Most bismuth compounds used in cosmetics have low toxicity when ingested, but they may cause allergic reactions when applied to skin.

“Matte lipsticks are heavy in wax and pigment but lighter in emollients. They have more texture than shine. Cremes are a balance of shine and texture. Glosses have a high shine and low color. Sheers and stains contain a lot of oil and a medium amount of wax with a tad of color. Shimmers have extra glimmer, which comes from mica or silica particles. Long-lasting color lipsticks contain silicone oil, which seals the color to your lips. Lip gloss usually comes in jars and contains different proportions of the same ingredients as lipstick but usually has less wax and more oil to make the lips shinier.”

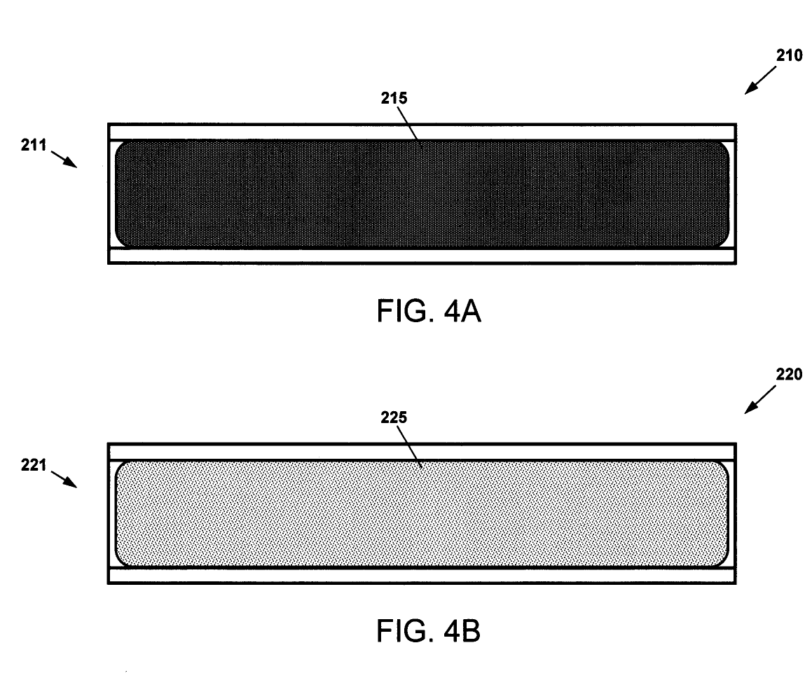

FIG. 4A is a cross-sectional view of a one halloysite tubule that may be incorporated into a lipstick formulation wherein the active agent in the tubule includes a colorant. FIG. 4B is a cross-sectional view of a one halloysite tubule that may be incorporated into a lipstick formulation wherein the active agent in the tubule includes a skin moisturizer. Referring to FIG. 4A, filled nanotubule 210 comprises an inorganic tubule 211 that preferably consists essentially of halloysite clay, and has substantially the same structure as was recited herein for tubule 110 of FIGS. 1 and 2. Referring to FIG. 4B, and in like manner, filled nanotubule 220 comprises an inorganic tubule 221 that preferably consists essentially of halloysite clay, and has substantially the same structure as was recited herein for tubule 110 of FIGS. 1 and 2.

Inorganic tubule 211 of FIG. 4A contains an admixture 215 for the purpose of providing or replenishing colorant into a lipstick formulation in which a plurality of such tubules 211 are dispersed. In one embodiment, tubule 211 may contain an admixture 215 comprising the formula disclosed at page 330 of the aforementioned “CDCl” of R. Winter, “castor oil, about 65 percent; beeswax, 15 percent; carnauba wax, 10 percent; lanolin, 5 percent; certified dyes, soluble; color lakes, insoluble; and perfume.” It will be apparent that the relative proportions of such ingredients may vary more or less, the main ingredients being about 55-75 percent castor oil, about 10-20 percent beeswax, about 5-15 percent carnauba wax, and about 2-10 percent lanolin.

Suitable soluble dyes may include but are not limited to those mentioned above; D & C Red No. 21 “Solvent Red 43. Tetrabromofluorescein. Classed chemically as a flouran color. A bluish pink stain . . . Insoluble in water but used to color oils, resins, and lacquers . . . The uncertified name for batches of this color is solvent Red 43.” (“CDCl” page 184); D & C Red No. 27, also a flouran color, is “Solvent Red 48. Philoxine B. Veri Pink. A xanthene dye.” (“CDCl” page 184); and D & C Red No. 34 Calcium Lake “Insoluble pigment prepared from D & C Red No. 34.” Red No. 34 is also known as “Fanchon Maroon. Deep Maroon. Classed chemically monoazo color . . . . The uncertified name for this color is Pigment Red 63:1.” (“CDCl” page 185.) Lakes are coloring ingredients “in which the dyes have been mixed with alumina hydrate to make them insoluble.” (“CDCl” page 168.)

In another embodiment, the coloring formula 215 within the nanotubules may be semi-permanent in nature and therefore may contain such ingredients as Isodecane, Trimethylsiloxysilicate, Dimethicone, Quaternium-18 Hectorite, and Propylparaben; It may also contain some combination of the following: Mica, Titanium Dioxide, Iron oxides, Red 30 Lake, Yellow 5 Lake, Blue 1 Lake, Carmine, Yellow 6 Lake, and Red 7 Lake, as found in the semi-permanent “color base coat” of MaxFactor's Lipfinity Everlites, Color #40, Enduring Immuable.

In another embodiment, the halloysite tubule admixture contains texture ingredients, such as frosting ingredients or those causing shimmer. As mentioned previously, a frosted appearance is due to pearlizing ingredients, including but not limited to bismuth oxychloride, bismuth subcarbonate and other bismuth compounds. Shimmer or shine agents include but are not limited to mica and silica particles.

Inorganic tubule 221 of FIG. 4B contains an moisturizer active agent 225 for the purpose of moisturizing skin on which such lipstick is applied. In one embodiment, tubule 221 may contain an admixture 225 comprising a combination of ingredients such as Sucrose Polycottonseedate, Brassica Campestris/Aleurites Fordi Oil Copolymer, Ozokerite, Beeswax (Cera Alba), Tocopheryl Acetate, Tocopherol, Ethylene Brassylate, Propylparaben, Propyl Gallate, Cydopentasiloxane, as is contained in the “moisturizing top coat” of MaxFactor's Lipfinity Everlites, Color #40, Enduring Immuable.

In another embodiment tubule 221 contains alternative active agents 225, such as a sunscreen agent that may be comprised of zinc oxide, avobenzene (butyl methoxydibenzoylmethane), titanium dioxide, and/or Mexoryl SX.

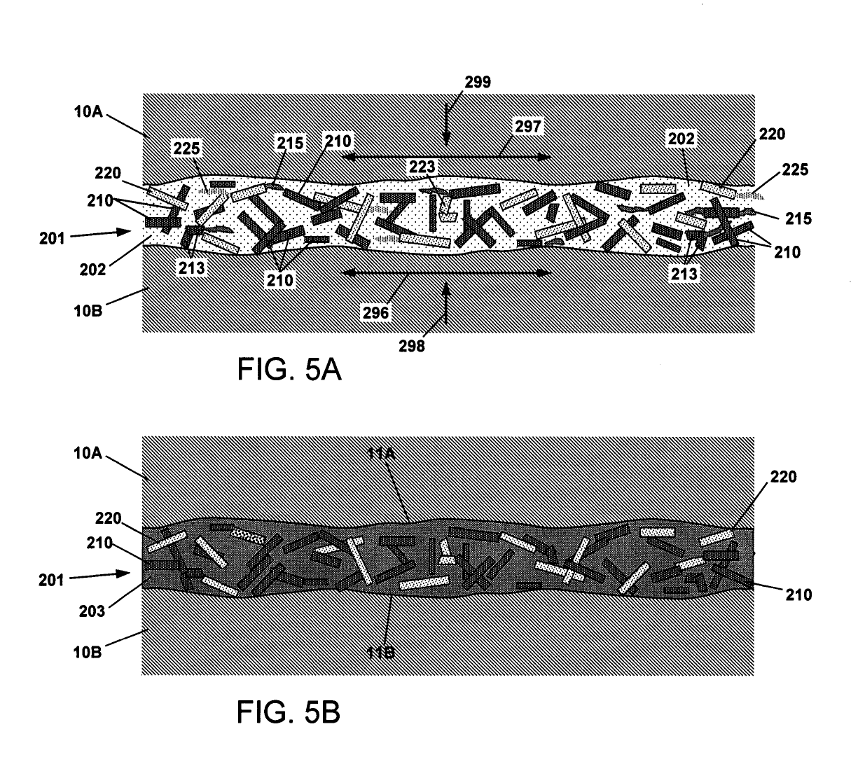

FIGS. 5A and 5B are schematic representations of the use of a lipstick formulation containing halloysite tubules loaded with an admixture, preferably including a colorant. Referring to FIG. 5A, lipstick formulation 201 is applied to each of lips 10A and 10B from a typical tubular applicator. (It is noted that such application step is not shown in FIG. 5A or 5B.) At some point in time, the coloration of the lipstick has faded, and it is desired to “replenish” the color, or the presence of other beneficial ingredients of the lipstick. The user compresses lips 10A and 10B together as indicated by arrows 299 and 298 and the user moves lips 10A and 10B laterally back and forth as indicated by bidirectional arrows 297 and 296. This motion provides compression and shearing forces on the thin film of lipstick 201 between lips 10A and 10B.

Lipstick 201 is comprised of nanotubules 210 that contain the colorant admixture 215. The compressive and shearing forces result in the discharge of colorant 215 into the bulk lipstick fluid 202, which provides lips 10A and 10B with their colored appearance. Without wishing to be bound to any particular theory, the applicant believes that the mechanism of release is a combination of desorption of the colorant from within the tubules due to the existence of a chemical concentration gradient as the original lipstick application is depleted; the mechanical pressure of the compressive action; fracture of some portion of the tubules (e.g. tubules 213); and the additional heat caused by pursing and shearing lips 10A and 10B together. This pursing causes friction and reduces convective heat transfer by exposure to air. Each effect in turn results in an increase in the temperature of the lipstick film 201, increasing the rate of discharge of colorant 215 from tubules 210/213; and a lowering of the viscosity of the bulk lipstick fluid 202. The discharged colorant 215 is thus also blended into the bulk lipstick fluid 202 by the motion of lips 10A and 10B. (For the sake of simplicity of illustration, in FIG. 5A there is only shown discharge of colorant 215 from some of the tubules 210, with it being understood that all of tubules 211 in lipstick film 201 may be discharging colorant to some extent into bulk fluid 202.)

Depending on the application, the extent of the desired release of colorant may be a relatively small or relatively large amount to effect a replenishing of the lipstick. The degree of release of colorant may be controlled by a) the concentration of filled tubules 210 contained in the lipstick 201; b) the size distribution of the tubules, i.e. larger tubules will carry more colorant and secrete more colorant per stimulation; and c) the composition of the tubules as relates to stiffness and resistance to fracture.

Referring to FIG. 5B, it can be seen at the conclusion of the compressive and shearing motion of lips 10A and 10B, the lipstick film 201 is comprised of a bulk fluid 203 that is higher in concentration of colorant 215, as indicated schematically by a the darker shading therein.

In preparing a lipstick formulation containing filled halloysite tubules, the filled halloysite tubules are mixed with the bulk lipstick at an elevated temperature such that the bulk lipstick becomes less viscous, thereby enabling the filled tubules to be mixed into the bulk. The tubules become at least partially filled with lipstick colorant by diffusion and are then mixed back into the bulk lipstick sample, with subsequent cooling to a semi-solid state suitable for placement into tubular lipstick containers. The remaining processing of the lipstick product would follow standard industry practice.

As is disclosed in the aforementioned publication of R. Johnson, Chemical and Engineering News 77 (28) Pg. 31 Nov. 12, 99), “Making lipstick is similar to making crayons—a lot of heating and mixing and stirring goes on. Simply put, the mixture is finely ground, and the waxes are added for texture and to maintain stiffness. Oils and lanolin are added for specific formula requirements. The hot liquid is then poured into cold metal molds where it solidifies and is further chilled. The formed lipstick is put through a flame for about half a second to create a smooth and glossy finish and to remove imperfections.” Reference may also be had to U.S. Pat. No. 6,022,209, “Device for Making Lipstick,” the disclosure of which is incorporated herein by reference.

Referring again to FIG. 5A, and in a further embodiment, lipstick film 201 may further comprise a portion of nanotubules 220 containing a moisturizer 225 (as described previously with reference to FIG. 4B) that is also dispersed by the shearing and compressive action of lips 10A and 10B. The effect of such moisturizer is to provide relief from dryness and chapping of the surfaces 11A and 11B of lips 10A and 10B.

Another facial make-up product that may be improved from the incorporation of halloysite tubules is mascara, where halloysite, filled or unfilled, can be used to increase the appearance of the eyelash length and volume without the “caked-on” look. By incorporating longer halloysite tubules (lengths ranging from 1 μm to 80 μm) into current mascara formulas, the length of the eyelash can be increased as the brush used to apply the mascara preferentially aligns the outer tubules along the length of the eyelash. Since the tubules are stiff and are adhered together by the mascara, they provide a support structure for the pigment on the eyelash. The halloysite tubules may be unfilled or they may be filled by mascara formulation or specific low molecular weight polymers to aid in forming an association between the carrier mascara formulation and the halloysite tubule.

In this embodiment a suitable mascara formulation includes but is not limited to a salt of stearic acid with pigments and/or lanolin, paraffin, and carnauba wax. As is disclosed in the aforementioned “CDCl” of R. Winter, stearic acid occurs naturally in butter acids, tallow, cascarilla bark, and other animal fats and oils. Lanolin, also known as Wool Fat or Wool Wax, is “a product of the oil glands of sheep . . . . A natural emulsifier, it absorbs and holds water to the skin. Chemically a wax instead of a fat. Contains about 25 to 30 percent water.” (“CDCl” page 319) Paraffin is “Obtained from the distillate of wood, coal, petroleum, or shale oil. Colorless or white, odorless, greasy, and not digestible or absorbable in the intestines. Pure paraffin is harmless to the skin . . . . Also used for extracting perfumes from flowers.” (“CDCl” page 386.) Carnauba wax or Copeinicia cerifer consists of “the exudates from the leaves of the Brazilian wax palm tree used as a texturizer” (“CDCl” page 136.)

The commercial product “Maybelline Illegal Lengths Mascara, Brownish Black, washable,” is an example of mascara meant to lengthen the appearance of eye lashes. This product comes in a small cylindrical tube with a straight brush. This mascara product may serve as a suitable carrier formula for the present invention. As is disclosed on the packaging of this product, the ingredients include water, acrylates copolymer, alcohol Denat, Beeswax, cyclopentasiloxane, glyceryl stearate, carnauba wax, PVP/Eicosene copolymer, stearic acid, paraffin, polyethylene, propylene glycol, polymethyl methacrylate, triethanolamine, PVP, silica, dimethiconol, methylparaben, hydroxyethylcellulose, imidazolidinyl urea, isopropyl titanium triisostearate, simethicone, butylparaben; and the product may contain various colorants. Other suitable mascaras include but are not limited to Maybelline Lash Discovery and Maybelline Full 'N Soft.

The formulations of these products are described in U.S. Pat. Nos. 4,871,536, 5,618,523, 6,015,574, 6,464,967, and 6,221,389. Brushes for the application of these products and methods for making such brushes are described in U.S. Pat. Nos. 4,887,622, 5,133,590, and 5,462,798. The disclosures of each of these United States patents are incorporated herein by reference.

In one embodiment, halloysite tubules may be filled with a suitable polymer to increase the appearance of length and/or the incorporation of the tubule into the mascara formulation. Suitable polymer fillers include but are not limited to anilane microcellulose (more specifically hydroxyethylcellulose), acrylic resin (also known as acrylate polymers) which are also used for waterproofing, Polyvinypyrrolidone (PVP) and its copolymers, and_Poloxamer 188 or Poloxane, which is “a liquid, nonionic, surfactant polymer. If chain lengths of polyoxyethylene and polyoxypropylene are increased, the product changes from liquid to paste to solid.” (“CDCl” page 410).

Reference may also be had to method for filling tubules with polymers, which is disclosed in the publication, “Pharmaceutical applications of size reduced grades of surfactant co-processed microcrystalline cellulose,” S. R. Levis et al, Int J. Pharm. 230, 25-33, 2001.

The filled halloysite tubules may then be incorporated into the process for making the mascara formulation. One such process is disclosed in United States Published Patent Application 2004/0141932, the disclosure of which is incorporated herein by reference.

Numerous other embodiments of the present invention are comprehended wherein a cosmetic formulation that is applied to skin contains nanotubules containing an active agent, such active agent being released and/or replenished into the bulk by time release, and/or compressive and/or shearing action along the skin surface. Such embodiments include replenishing moisturizer, replenishing sunscreen, skin balancers, anti-wrinkle creams, smoothing lotions, anti-cellulite lotions, longer lasting perfumes, anti-itch creams, anti-bacterial hand lotions, acne medicated lotions, vitamin E topical treatments.

In an embodiment where halloysite tubes are filled with sunscreen lotion, suntan lotion, or suntan oil, the halloysite tubes may be loaded with the active UVA/UVB blocker agent with or without a carrier lotion, a skin repair agent, a skin colorant or color fixative in the case of a suntan lotion, or the any combination of ingredients from the full sunscreen formulation, including the full formulation. Sunscreen filled halloysite tubules may also be mixed into formulations for other cosmetic products where a sunscreen component is desirable for the protection of skin. Such cosmetics include but are not limited to moisturizers, foundations, translucent and pressed powders, body wash, soaps, creams, lotions, ointments, medicated ointments, and facial cleansers.

It is well known that exposure to the sun causes skin damage. The measure of protection provided by any sunscreen product is summarized as the SPF or Sun Protection Factor. As is disclosed at pp. 500-502 in the aforementioned “CDCl” of R. Winter, “The SPF number is based on the time it would take you to first turn slightly red from the sun. That is if your skin begins to turn pink after ten minutes, a product with SPF 15 would theoretically keep you from sunburning for 150 minutes under ultraviolet radiation . . . . SPF 30 does not give twice the protection of SPF 15. SPF 15 absorbs 93 percent of the sunburning rays, and SPF 30 absorbs 97 percent.”

As is disclosed in Photodermatology, Photoimmunology & Photomedicine, February 2001, at pages 2-10, “The SPF number tells you only about how long a product will protect you from getting a sunburn, protecting your skin from the sun's UVB rays. Surprisingly, it doesn't provide any information about the Sun's UVA rays. The sun's UVA rays have no apparent, immediate impact on skin, but they are thought to be the cause of wrinkles, hyperpigmentation, and skin cancer.”

As is disclosed in the Journal of Investigative Dermatology, November 2001, at pages 1186-1192, “UVA radiation is the primary cause of skin cancer . . . while the typical SPF 15 sunscreen formulation protected against 97% of UVB radiation, it kept out only a limited amount of UVA radiation.”

As is disclosed in Photodermatology, Photoimmunology & Photomedicine, December 2000, at pages 250-255), “[For protection against UVA] look for one of the following ingredients: avobenzone, titanium dioxide, zinc oxide, and (outside of the United States) Mexoryl SX;” and in the aforementioned “Beauty Bible,” at pp. 142-143, “Mexoryl SX and avobenzene can be used in conjunction with other sunscreen ingredients.”

Skin damage is not limited to the uncomfortable burn and accompanying redness. In addition to the damage that eventually occurs on the surface of the skin, such skin contains components that are central to the immune system of the body. The Langerhans cells in the epidermis prevent bacteria from attacking the system and prevent cell mutation. A few minutes of unprotected sun exposure can damage the Langerhans cells for a prolonged period of time, on the order of weeks. In addition to damaging the immune system, the sun also directly attacks the collagen structure of the skin, changing it from a cohesive network of support into a disorderly, weakened mass. It also thickens the exterior of the skin, chokes off the skin's blood supply of the skin, and reduces the elasticity of the skin.

For these reasons, many types of skin care and cosmetic products are increasingly including broad spectrum sunscreen ingredients in their formulations. Broad spectrum protection refers to protection against both UVA and UVB wavelengths of sunlight. According to “Skin Therapy Letter” published by the Division of Dermatology at the University of British Columbia (1997, volume 2, number 5, “Update on Sunscreens”) the “UVA [range is] 315 [through] 400 nanometers.” (And according to the United States Food and Drug Administration, the UVB range is from 280 to 315 nanometers.) The range of protection for the following sunscreen ingredients is listed as “Padimate O, 210-315 nanometers; Benzophenones, 250-350 nanometers; Octyl methoxycinnamate, 290-320 nanometers; Avobenzone, 320-400 nanometers; Titanium dioxide, 290-700 nanometers; and Zinc oxide 290-700 nanometers.”

In addition to the ingredients mentioned previously, such sunscreen preparations may also contain alcohol, p-aminobenzoic acid and derivatives, benzyl salicylate, cinnamic acid derivatives, and coumarin.

P-aminobenzoic Acid or para-aminobenzoic Acid is a “colorless or yellowish acid found in vitamin B complex. In an alcohol and water solution plus a light perfume, it is sold under a wide variety of names as a sunscreen lotion to prevent skin damage. It is also sold as a local anesthetic in sunburn products. It is used medicinally to treat arthritis. However, it can cause allergic eczema and a sensitivity to light in susceptible people whose skin may react to sunlight by erupting with a rash, sloughing, and/or swelling.” (“CDCl” page 385.) Benzyl salicylate, one of the salicyclic acid salts mentioned previously, acts as a “fixative in perfumes and a solvent in sunscreen lotions. It is a thick liquid with a light, pleasant odor, and is mixed with alcohol or ether.” (“CDCl” page 104.)

Cinnamic acid is “Used in suntan lotions and perfumes. Occurs in storax, Balsam Peru, cinnamon leaves, and coca leaves. Usually isolated from woodrotting fungus.” (“CDCl” page 155.) Coumarins, as known as tonka bean or cumarin, are a “fragrant ingredient of tonka beans, sweet woodruff, cramp bark, and many other plants. It is made synthetically as well. Used in over three hundred products in the

United States including acne preparations, antiseptics, deodorants, “skin fresheners”, hair dyes, and shampoos. It has been widely used as a fragrance in soaps, detergents, perfumes, and sunscreens . . . It has anti-blood clotting effects and anticlotting ingredients are derived from it.” (“CDCl” page 175.) Oxybenzone is “derived from isopropanol, it is used as a sunscreening ingredient.” (“CDCl” page 381.) Isopropyl alcohol, commonly known as isopropanol, is an “antibacterial, solvent, and denaturant. Solvent for the spice oleoresins . . . . It is prepared from propylene, which is obtained in the cracking of petroleum.” (“CDCl” page 306.)

In a further embodiment, the halloysite tubules of the present invention may contain active sun blocking agents and optionally inactive carrier ingredients. Suitable sun blocking materials include but are not limited to UVA blockers such as zinc oxide, titanium dioxide, avobenzene which is also known as butyl methoxydibenzoylmethane or Parsol 1789, and Mexoryl SX, and UVB blockers such as_para-aminobenzoic acid, salicylates, and oxybenzone. Suitable carrier ingredients are listed previously herein.

In one embodiment, halloysite tubules may contain a suntan cream or emulsion which may comprise, but is not limited to, the following ingredients as is disclosed at pp. 500-502 of the aforementioned “CDCl” of R. Winter: “para-aminobenzoic acid, mineral oil, sorbitan stearate, poloxamers, and 62 percent water.” Reference may also be had to U.S. Pat. No. 5,980,871, “Sunscreen compositions,” of Lukenbach et al, the disclosure of which is incorporated herein by reference.

Para-aminobenzoic acid is a “colorless or yellowish acid found in vitamin B complex. In an alcohol and water solution plus a light perfume, it is sold under a wide variety of names as a sunscreen lotion to prevent skin damage. It is also used as a local anesthetic in sunburn products. It is used medicinally to treat arthritis.” (“CDCl” page 385) Mineral Oil or “White Oil. Widely used . . . . A cosmetic lubricant, protective ingredient, and binder. It is a mixture of refined liquid hydrocarbons derived from petroleum. Colorless, transparent, odorless, and tasteless. When heated, it smells like petroleum. It stays on top of the skin and leaves a shiny protective surface.” (“CDCl” page 354) Sorbitan Stearate also known as “Sorbitan Monostearate. An emulsifier in cosmetics creams and lotions, a solubilizer of essential oils in water . . . . Manufactured by reacting edible commercial stearic acid with sorbitol.” Sorbitan Tristearate can be used as an alternative. (“CDCl” page) Poloxamer 188 or “Poloxane. A liquid, nonionic, surfactant polymer. If chain lengths of polyoxyethylene and polyoxypropylene are increased, the product changes from liquid to paste to solid.” (“CDCl” page 410).

In another embodiment, the halloysite tubules of the present invention may contain a suntan ointment. Such suntan ointment may contain petrolatum, stearyl alcohol, mineral oil, sesame oil, and calcium stearate.

Petrolatum, also known as Vaseline, petroleum jelly, and paraffin Jelly, “is a purified mixture of semisolid hydrocarbons from petroleum. Yellowish to light amber or white, semisolid unctuous mass, practically odorless and tasteless, almost insoluble in water . . . . it makes creams smoother. Helps to soften and smooth the skin in the same way as any other emollient and is less expensive. The oily film helps prevent evaporation of moisture from the skin and protects the skin from irritation.” (“CDCl” page 397.) Stearyl Alcohol, or Stenol is “A mixture of solid alcohols prepared from sperm whale oil. Unctuous, white flakes, insoluble in water, soluble in alcohol and ether. Can be prepared from sperm whale oil. A substitute for cetyl alcohol to obtain a firmer product at ordinary temperatures. Used in pharmaceuticals, cosmetic creams, for emulsions, as an antifoam ingredient, and a lubricant” (“CDCl” page 493.) Sesame Oil, Sesamum indicum Oil, is “The edible seeds of an East Indian herb that has a rosy or white flower. The seeds . . . yield a pale yellow oil used in the manufacture of margarine. The oil has been used as a skin softener and contains elements active against lice. It is also used in hair conditioners.” (“CDCl” page 454.) Calcium Stereate is “Prepared from limewater, it is an emulsifier used in hair-grooming products. Also as a coloring ingredient, in water proofing, and in paints and in printing ink.” (“CDCl” page 128.)

In another embodiment, halloysite tubules of the present invention may contain a suntan lotion. Such a suntan lotion may contain e.g., methyl anthranilate, propylene glycol, ricinoleate, glycerin, and about 65 percent alcohol and about 10 percent water.

Methyl anthranilate is “Used as an ‘orange’ scent for ointments, in the manufacture of synthetic perfumes, and in suntan lotions. Occurs naturally in neroli, ylang-ylang, bergamot, jasmine, and other essential oils. Colorless to pale yellow liquid with a bluish fluorescence and a grapelike odor. It is made synthetically from coal tar.” (“CDCl” page 348.) Propylene Glycol or 1,2-Propanediol is “One of the most widely used cosmetic ingredients, it is a clear, colorless, viscous liquid, slightly bitter-tasting. It is the most common moisture-carrying vehicle other than water itself in cosmetics. It has better permeation through the skin than glycerin and is less expensive, although it has been linked to more sensitivity reactions. Absorbs moisture, acts as a solvent and a wetting agent . . . . Its use is being reduced, and it is being replaced by safer glycols such as butylenes and polyethylene glycol.” (“CDCl” page 428.) Ricinoleate is a “Salt of ricinoleic acid found in castor oil. Used in the manufacture of soaps.” (“CDCl” page 442.) Ricinoleic Acid is “A mixture of fatty oils found in the seeds of castor beans. Castor oil contains 80 to 85 percent ricinoleic acid. The oily liquid is used in soaps, added to Turkey-red oil, and used in contraceptive jellies. Also used in cosmetics as an emollient.” (“CDCl” page 442.) Salicylates, the amyl, phenyl, benzyl, methyl, glyceryl, dipropylene, glycol esters, salts of salicylic acid, are made from “a number of foods that naturally contain salicylate, such as almonds, apples, apple cider, apricots, blackberries, boysenberries, cherries, cloves, cucumbers, currants, gooseberries, grapes, nectarines . . . . The salts are used as sunburn preventatives and antiseptics.” (“CDCl” page 448.)

Salicylic acid or methyl salicylate, which is the base of the salicylcates, “Occurs naturally in wintergreen leaves, sweet birch, and other plants. Synthetically prepared by heating phenol with carbon dioxide. It has a sweetish taste and is used as a preservative and antimicrobial. Used externally as an antiseptic ingredient, fungicide, and skin sloughing ingredient. It is used as a preservative and antimicrobial at 0.1 to 0.5 in skin softeners, face masks, hair tonics, deodorants, dandruff preparations, protective creams, hair dye removers, and suntan lotions and oils. It is antipruritic (anti-itch). It is also used in making aspirin. It is used as a keratolytic drug applied topically to treat acne to slough the skin. Being widely promoted at this writing in anti-aging betahydroxide products . . . . It is being substituted for the ‘alpha’ in alpha-hydroxide products because it is said to be less irritating to the skin. The European Union has banned it for children under three years except in shampoos. It can be absorbed through the skin.” (“CDCl” page 448.)

In another embodiment, halloysite tubules of the present invention may contain a suntan oil. Such suntan oil may contain e.g., salicylates, about 40 percent sesame oil, and about 55 percent mineral oil. One common suntan oil formula disclosed at pp. 500-502 in the aforementioned “CDCl” includes 2-ethyl hexyl salicylate, 5 percent; sesame oil, 40 percent; mineral oil, about 55 percent; perfume; color; and an antioxidant.

In a further embodiment, halloysite tubules contain ingredients to repair sun damage. Such repair ingredients include but are not limited to methyl salicylate, tretinoin (Retin-A or Renova), Coenzyme Q10 (COQ10), allantoin, and zinc oxide.

In yet another embodiment of the present invention, halloysite tubules may contain ingredients to increase the appearance of skin pigmentation, i.e. a darkening of the skin. Such ingredients include but are not limited to Psoralen®. Psoralen® is “named for the Latin psora, meaning itch, and derived from a plant. Used in the treatment of vitiligo (lack of skin pigment), in sunscreen to increase tanning, and in perfumes.” (“CDCl” page 431.) In this embodiment, the object is not necessarily to prevent sun damage but to artificially induce a coloring of the skin, commonly referred to as a suntan. There are many such products currently being sold as “sunless tanning” lotions. Common objections to such products by their users include a streaky appearance from uneven application, and an orange hue that occurs when too much cream is used in any one place. According to the present invention, in such a cream, the halloysite tubule is filled with an active coloring ingredient and dispersed within a lotion as occurs under ordinary mixing conditions; when this formulation is applied to the skin, it enables a more even, natural appearance of a “tan,” as the tubules distribute and slowly release the active ingredient. It may also be advantageous to fill the tubule with the complete “sunless tanning” formulation in order to “slow release” the formula, thereby replenishing the tan and allowing the tanning effect to last longer.

In accordance with the above embodiment, the halloysite tubules may be filled with an active coloring agent. Such coloring ingredients include but are not limited to dihydroxyacetone. As is disclosed in the aforementioned “CDCl,” dihydroxyacetone is “also used as an emulsifier, humectant, and fungicide . . . A white powder that turns colorless in liquid form, it colors the skin an orange-brown shade, giving it a suntanned appearance. It is an ingredient in some suntan lotions for use indoors without sunlight. Obtained by the action of certain bacteria on glycerol, it has a sweet taste and a characteristic odor.”

A suitable carrier lotion for such coloring agents may be a suntan ointment. Such a suntan ointment may be comprised of petrolatum, stearyl alcohol, mineral oil, sesame oil, and calcium stearate.

As many cosmetics products are incorporating sun-protection ingredients into their formulas, other suitable carrier fluids or powders include, but are not limited to foundation, concealer, moisturizer, body lotion, ointment, face cream, cleanser, lipstick, translucent or balancing powder, and medicated ointments. Sun protection is of particular importance where the active ingredient in a medicated formulation where the active ingredients can cause light sensitivity. Formulations for these various carrier media are described elsewhere in this specification.

In a similar manner, halloysite tubes may provide continual slow release or timed release of active ingredients in an lotion for the treatment of acne. Acne is a condition where “skin pores become plugged with an oily substance, sebum, and other materials such as pigment, dead cells, and bacteria. If the plug (comedo) remains just below the surface, it appears as a very small, round, whitish bump, a ‘whitehead.’ If it reaches the skin surface, it looks like a black dot, a ‘blackhead.’ In some cases, the plugged pore may burst, thereby releasing its oily contents into the surrounding tissue and causing inflammation. This results in the formation of pimples, pus-filled lesions, or even cysts, cavities containing a sticky fluid.” (“CDCl” page 50.) Halloysite tubules filled with active ingredients such as antibacterials, wound healing ingredients, and acne specific medications may be used for continual treatment of the acne to better heal the skin and prevent scarring.

In one embodiment, the halloysite tubules contain acne specific medication including but not limited to Clenia with active ingredients of 5 percent sulfur and 10 percent sodium sulfacetamide or Neutragena oil-free Acne Wash with 2 percent salicyclic acid. Such medication is disclosed at page 602 with regard to acne medication in Skin Disease Diagnosis and Treatment, 2^{nd} Ed., Thomas P. Habif, et al., Elsevier Mosby, 2005 Alternatively, one may apply tubules filled with Retin-A, “a prescription drug for the treatment of acne, it is a vitamin A derivative . . . . It reportedly plumps the skin, smoothes fine wrinkles, and begins to reverse other less visible signs of sun damage. The drug is believed to increase cell turnover, so dull surface cells are shed more quickly. It thickens the epidermis and improves texture, elasticity, and blood circulation.” (“CDCl” page 440.) Such tubules may further include the active ingredient tretinoin.

In one embodiment the halloysite tubules may contain a more general skin repair ingredient such as Methyl salicylate, Coenzyme Q10 (COQ10), Allantoin, and zinc oxide. Salicylic acid or methyl salicylate, “occurs naturally in wintergreen leaves, sweet birch, and other plants. Synthetically prepared by heating phenol with carbon dioxide. It has a sweetish taste and is used as a preservative and antimicrobial. Used externally as an antiseptic ingredient, fungicide, and skin sloughing ingredient. It is used as a preservative and antimicrobial at 0.1 to 0.5 in skin softeners, face masks, hair tonics, deodorants, dandruff preparations, protective creams, hair dye removers, and suntan lotions and oils. It is antipruritic (anti-itch). It is used as a keratolytic drug applied topically to treat acne to slough the skin. Being widely promoted at this writing in anti-aging betahydroxide products . . . . It is being substituted for the ‘alpha’ in alpha-hydroxide products because it is said to be less irritating to the skin.” (“CDCl” page 448.)

COQ10, as known as Coenzyme Q10 or Ubiquinone, is “an antioxidant nutrient . . . It is being added to cosmetics to scavenge free radicals and ‘fight signs of aging skin.’” (“CDCl” page 172). Allantoin is “Used . . . to help heal wounds and skin ulcers and to stimulate the growth of healthy tissue. Colorless crystals, soluble in hot water, it is prepared synthetically by the oxidation of uric acid or by heating uric acid with dichloroacetic acid.” (“CDCl” page 58) Zinc oxide or “Flowers of Zinc” is “a creamy white ointment used medicinally as an astringent, antiseptic, and protective in skin diseases. Zinc is believed to encourage healing of skin disorders.” (“CDCl” page 552)

In one embodiment, halloysite tubules contain anti-bacterial or germicidal ingredients including but not limited to bithionol, tribromsalan, hydroxyethyl isobutyl piperidine carboxylate, and zinc dibutyldithiocarbamate.

Suitable Carrier fluids for both the active ingredients listed above as well as for the halloysite tubules include but are not limited to a cleansing lotion. Suitable cleansing lotions are described at page 158 of the aforementioned “CDCl” as being “used to dissolve sebum, loosen particles of grime, and to facilitate the removal of dirt. They usually contain mineral oil, triethanolamine stearate, and water. Among other ingredients commonly used are alcohol, alkanoamines, allantoins, antibacterials, and preservatives, methyl and propyl parabens, fatty alcohols, lanolin, perfumes, glycerol, propylene glycol, fatty oils, thickeners, and waxes. A well-known hypoallergenic cold cream contains water, mineral oil, waxes, borax, and depollenixed beeswax.”

Another suitable carrier fluid would be a liquefying cream, which is a cleansing cream designed to liquefy when rubbed into the skin. Such a cream usually contains paraffin, a wax, stearic acid, sodium borate, liquid petrolatum of about 50-60 weight percent, and water at about 20-30 weight percent. Another suitable carrier fluid may be comprised of a moisturizer, a foundation, a concealer, and or an exfoliating medium such as a facial scrub. The formulations of these ingredients are described elsewhere in this specification.

In another embodiment, there is provided a hybrid powder for skin balancing that is comprised of halloysite tubules filled with active agents. It is noted that most people have “combination skin”, meaning that some facial areas are oily (particularly the forehead, nose, and chin) and others are dry. Finding a balance of moisturizing and cleansing is extremely difficult and with the harsh chemicals often present in the ambient atmosphere, skin protection is very desirable. It is also noted that many women wear foundation to smooth out the appearance of the skin. Unfortunately, foundation generally clogs pores and is often obvious, creating a caked on look.

In this embodiment, there is provided a powder comprised of halloysite, wherein such powder contains a large percentage of tubules, i.e. between about 0.10 and about 20 weight percent. At least about 2 percent of the tubules are filled with an oil based or emulsion based moisturizer such as e.g. propylene glycol, sesame oil, mineral oil, and petrolatum. When such powder is applied to the skin, the powder itself acts to absorb oils and dry oily skin, while the filled tubules release moisturizer to treat dry skin. In a further embodiment, the powder and tubules may also be dyed to match various skin tones and may be provided in medicated varieties that release anti-wrinkle agents, anti-bacterial agents, anti-acne, and/or insect repellent agents as well as moisturizers and perfumes.

Anti-wrinkle agents include those such as tretinoin (also known as the active ingredient in Renova, as Retin A or Renova 0.05 percent emolient), Allantoin, Coenzyme Q10, bovine serum albumin., and methyl salicylate. Commercial anti-wrinkle creams include Avage, a cream containing 0.1 percent Tazarotene as the active ingredient, as is disclosed at page 603 of Skin Disease Diagnosis and Treatment, 2^{nd} Ed., Thomas P. Habif, et al. Elsevier Mosby, 2005.

Tretinoin is also referred to as Vitamin A Retinoic Acid or Retin-A. Retin-A is “a prescription drug for the treatment of acne, it is a vitamin A derivative that reportedly plumps the skin, smoothes fine wrinkles, and begins to reverse other less visible signs of sun damage. The drug is believed to increase cell turnover, so dull surface cells are shed more quickly. It thickens the epidermis and improves texture, elasticity, and blood circulation. “(“CDCl” page 440.) Allantoin is “Used . . . to help heal wounds and skin ulcers and to stimulate the growth of healthy tissue. Colorless crystals, soluble in hot water, it is prepared synthetically by the oxidation of uric acid or by heating uric acid with dichloroacetic acid.” (“CDCl” page 58) COQ10 or Coenzyme Q10 or Ubiquinone is “An antioxidant nutrient . . . It is being added to cosmetics to scavenge free radicals and ‘fight signs of aging skin’” (“CDCl” page 172). Bovine serum albumin (BSA) is used as a skin tightener to make skin feel firmer. (“CDCl” page 461) Methyl salicylate, which is the base of the salicylcates, “Occurs naturally in wintergreen leaves, sweet birch, and other plants. Synthetically prepared by heating phenol with carbon dioxide. It is used as a preservative and antimicrobial at 0.1 to 0.5 percent (?) in skin softeners, face masks, hair tonics, deodorants, dandruff preparations, protective creams, hair dye removers, and suntan lotions and oils. It is antipruritic (anti-itch). It is used as a keratolytic drug applied topically to treat acne to slough the skin. Being widely promoted at this writing in anti-aging betahydroxide products . . . . It is being substituted for the ‘alpha’ in alpha-hydroxide products because it is said to be less irritating to the skin. It can be absorbed through the skin.” (“CDCl” page 448)

Ingredients to combat acne are very similar to those used to decrease wrinkles, as the goal with acne treatment is to increase the sloughing of the skin, while promoting healthy new skin growth. Therefore, halloysite tubules in this embodiment may contain tretinoin (also known as Retin A or Renova), Allantoin, thioglycerol, and zinc oxide. Thioglycerol and zinc oxide have been described previously in this specification.

In another embodiment, the goal is not to reduce the actual wrinkling of skin, but to fill such wrinkles in order to reduce their appearance. In such an embodiment a halloysite tubule may contain filling agents including but not limited to hyaluronic acid and collagen.

Hyaluronic acid is “a sugar compound present in all connective tissue in vertebrates. In humans, it is found in high concentrations in the skin, cartilage, in the umbilical cord, testes, and in synovial fluid. In gel form, hyaluronic acid binds to water and provides volume to easily fill in larger folds of the skin around the mouth and cheeks. A non-animal form of hyaluronic acid has already been approved for use as a wrinkle filling agent in Canada, Europe, and Australia. If there is a lot of swelling, cosmetics containing this ingredient may help because it absorbs moisture.” (“CDCl” page 287) Collagen is a “Protein substance in connective tissue. In cosmetics, it is usually derived from animal tissue. The collagen fibers in connective tissues of the skin undergo changes from aging and overexposure to the sun that contribute to the appearance of wrinkles and other outward signs of aging . . . It is being used to fill out acne scars and other depressions, including wrinkles, by injection . . . Derived from animal skin and ground-up chicken feet.” (“CDCl” page 165) Collagen amino acids are “The major protein of the white fibers of connective tissue . . . insoluble in water, but easily altered to gelatins by boiling in water, dilute acids, or alkalies.” (“CDCl” page 165)

In another embodiment the halloysite tubes may contain anti-bacterials, anti-fungals, anti-infectives, antibiotics, and/or germicides. Such tubules including the ingredients contained therein are preferably provided in powder form. The goal of such powders is to protect both the powder itself, as well as the person applying it, from infection by any of the agents such as bacteria, fungi, viruses, or other germs. In some instances, these medicated powders may also serve to treat already developed infections.

In one embodiment, halloysite tubules contain anti-bacterial, anti-fungal or germicide ingredients including but not limited to propylparaben, salicyclic acid, copper and zinc undecylenate, chlorobutanol, bithionol, tribromsalan, hydroxyethyl isobutyl piperidine carboxylate and zinc dibutyldithiocarbamate.

Propylparaben or “Propyl p-Hydroxybenzoate, “Developed in Europe, the esters of p-hydroxybenzoic acid are widely used in the cosmetic industry as preservatives and bacteria- and fungus-killers. They are active against a variety of organisms, are neutral, low in toxicity, slightly soluble, and active in all solutions, alkaline, neutral, or acid . . . . Used medicinally to treat fungus infections . . . Less toxic than benzoic or salicylic acid.” (CDCl Pg 429) Salicylic acid or “methyl salicylate has been described previously in this specification.

Fungicides include “Any substances that kill or inhibit the growth of fungi. Older types include a mixture of lime and sulfur, copper oxychloride, and Bordeaux mixture. Copper naphthenate has been used to impregnate textile fabrics used for tenting and military clothing. Copper undecylenate with zinc undecylenate is used in foot powders and sprays.” (“CDCl” page 252.) Chlorobutanol is a white, crystalline alcohol that is used as a more general germ killer (“CDCl” page 92).

Medicated formulations of antifungal filled halloysite tubules may include Nystatin, which is described in 2005 Mosby's Drug Consult, Elsevier Mosby pp. II-2145-21492005, Nystatin is also an active ingredient of formulations sold under brand names such as Bio-statin and Mycostatin. As is described in such reference, Nystatin, which has the empirical chemical formula C₄₇H₇₅NO₁₇ with a molecular weight of 926.11 “is an antifungal antibiotic which is both fungistatic and fungicidal in vitro against a wide variety of yeasts and yeast-like fungi . . . . Nystatin is a polyene antibiotic of undetermined structural formula that is obtained from streptomyces noursei” Topically, Nystatin comes in cream, topical powder, and ointment. It is also available in oral suspension, oral tablets, vaginal tablets, and pastilles, the latter being a relatively hard, jelly-like preparation made from a mixture of glycerin and gelatin, to which a medicament and flavorings may be added. In addition to medicaments, pastille ingredients may include e.g., anise oil, cinnamon oil, and/or sucrose.

One suitable ointment may include e.g., 100,000 units of Nystatin per gram in Plastibase (a plasticized hydrocarbon gel), a polyethylene and mineral oil gel base. A suitable topical powder may be comprised of 100,000 units of Nystatin per gram, distributed in talc. A suitable cream may be comprised of “100,000 units of Nystatin per gram in an aqueous, perfumed vanishing cream base containing aluminum hydroxide concentrated wet gel, titanium dioxide, propylene glycol, cetearyl alcohol (and) ceteareth-20, white petroleum, sorbitol solution, glycerol monostearate, polyethylene glycol monostearate, sorbic acid, and simethicone,” as is disclosed in the aforementioned Mosby's reference at page II-2146. As used herein, a “unit” of Nystatin is meant to indicate a USP unit, i.e. a United States Pharmacopoeia unit, which is in reference to the level of activity of the particular medicament.

In the present invention, halloysite tubules may contain the active ingredient, Nystatin only, or a combination of the above formulations of ointment, powder, or cream, as well as the formulations of the suspension and/or tablets mentioned above. The halloysite tubules containing only the Nystatin may also be distributed into the above carrier formulations.

In one embodiment, halloysite tubes are filled with antipruritic or anti-itch ingredients. These include but are not limited to methyl salicylate, described previously herein, and hydrocortisone, discussed below.

In one embodiment, halloysite tubules are filled with anti-inflammatory ingredients with or without carrier fluids. Such anti-inflammatory ingredients include but are not limited to hydrocortisone, pimecrolimus, and tacrolimus.

Hydrocortisone is “an adrenal gland corticosteroid hormone introduced in 1952, used to decrease severe inflammation, as an adjunctive treatment for ulcerative colitis, and proctitis, for shock, and to treat adrenal insufficiency. Also suppresses the immune response, stimulates bone marrow, and influences protein, fat, and carbohydrate metabolism.” (“CDCl” page 288) Hydrocortisone is chemically known as pregn-4-ene-3, 20-dione, 11,17,21-trihydroxy-, 11β and has a molecular weight of 362.46. Pimecrolimus, sold commercially as Elidel in 1 percent creams and Tacrolimus, sold commercially as Protopic in 0.03 percent creams are steroid free anti-inflammatories.

One important application of such an anti-inflammatory and/or anti-bacterial powder or lotion is to combat diaper dermatitis or “diaper rash” in infants and incontinent adults, and may also help prevent bed sores for bed-ridden patients. Diaper rash is caused by elevations in skin pH that occur when urine is mixed with stools, predisposing to the breakdown of the epidermal layer. Effective prevention requires highly absorbent disposable diapers that effectively draw moisture away from the skin.”

In one embodiment a powder contains a mixture of halloysite tubules, approximately 5 percent unfilled to absorb urine and sweat and wick these irritants away from skin, approximately 15 percent filled with an antibacterial ingredient as previously described herein, or a commercially available mupirocin (also known as bactroban) cream, which is described at page 222 of Skin Disease Diagnosis and Treatment, 2^{nd} Ed., Thomas P. Habif, et al. Elsevier Mosby, 2005; approximately 35 percent filled with hydrocortisone or other anti-inflammatory, and the remainder, approximately 45 percent filled with moisturizer ingredient such as petrolatum. Said powder may be applied during a diaper change or may be incorporated into the diaper itself such as petrolatum is incorporated into the diaper area of Pamper's Rash Guard Diapers, also described at page 222 of the Habif reference. The powder may also contain a significant portion of inactive ‘filler’ powder with ingredients such as talc, kaolin, zinc oxide, starch, magnesium carbonate, and sometimes boric acid, the common ingredients of baby powder. (“CDCl: page 92).

In another embodiment, the powder contains only one or a limited combination of the above tubules with or without filler powder.

In another embodiment, the above halloysite tubules are incorporated into a baby cream, baby lotion, baby oil, or baby soap, or the active ingredients are mixed with these as carrier fluids.

Baby cream formulations are “aimed at protecting babies' skin against irritation and soothing it, such formulas usually contain mineral oil, paraffin, lanolin, white beeswax, and ceresin. They may also contain many other ingredients, including petrolatum, mineral wax, glyceryl monostearate, methyl- and propylparabens, extract of lanolin, sterol, hydrogenated fatty oils, and spermaceti.” (“CDCl” page 92.) Similarly, Baby lotion formulations are “aimed at protecting, soothing, and cleansing the delicate skin of babies. Usually contains antimicrobials, emulsifiers, humectants to retain moisture, thickeners, and often some perfume. The product may also contain lanolin, mineral oil, cetyl alcohol, preservatives, and antioxidants.” (“CDCl” page 92.) Baby oil “Usually contains mineral oil, palmitate, lanolin, vegetable oils, and lanolin derivatives.” (“CDCl” page 92.) Baby soap is “Usually a mild sodium soap of coconut and/or palm oil. Some are made of polyunsaturated vegetable oils. Baby soaps may contain colloidal oatmeal, a mild, soap-free sudsing ingredient, lanolin derivatives, and a germ killer such as chlorobutanol.” (“CDCl” page 92.)

In one embodiment, halloysite tubules contain an insect repellent. Insect repellents are an example of a product where the long elution times characteristic of halloysite tubules are advantageous, as it limits the need for re-application and lowers the amount of active ingredient (and thus toxicity) of the effective dose. Active insect repellent ingredients include but are not limited to Diethyl Toluamide (DEET), phenoxyethanol, and citronella oil.

Diethyl Toluamide (DEET) is made from m-toluoyl chloride and diethylamine in benzene or ether, and is a liquid that is soluble in water.

Phenoxyethanol or 2-phenoxyethanol is an “oily liquid with a faint aromatic odor . . . Widely used as a fixative for perfumes, bactericide, insect repellent, and topical antiseptic.” (“CDCl” page 399.) Citronella Oil or Cymbopogon nardus, is “a natural food flavoring extract from fresh grass grown in Asia. Used in perfumes, toilet waters, and perfumed cosmetics; also as an insect repellent.” (“CDCl” page 157.)

An overall method for the preparation of hybrid powder for skin balancing that is comprised of halloysite tubules will now be described.

Such powders with properties such as anti-itch, anti-inflammatory, anti-microbial, and anti-wrinkle, may be incorporated through standard mixing processes into a cream or lotion.

In one embodiment such a lotion is a hand lotion. Such hand lotions are emollients, and most contain stearic acid, lanolin, and water. They may also contain cetyl alcohol, mineral oil, glycerin, potassium hydroxide, perfume, and glyceryl monostearate. Recent formulas may also contain healing ingredients such as allantoin and water-repellent silicones to protect the hands against further irritation from water, detergents, or wind. The pH level of most hand creams and lotions is between about 5 and about 8. A typical example of a hand cream contains cetyl alcohol, 2 percent; lanolin, 1 percent; mineral oil, 2 percent; stearic acid, 13 percent; glycerin, 12 percent; methylparaben, 0.15 percent; potassium hydroxide, 1 percent; water, 68 percent; and perfume in sufficient amounts. A typical example of a hand lotion contains cetyl alcohol, 0.5 percent; lanolin, 1 percent; stearic acid, 3 percent; glycerin, 2 percent; methylparaben, 0.1 percent; triethanoamine, 0.75 percent; water, 85 percent; and in some instances, a perfume.

In one embodiment, the lotion is a moisturizing lotion. Such moisturizing lotions are typically a mixture of oils. The most common ingredients of such lotions include mineral oil, stearic acid, lanolin, beeswax, sorbitol, and polysorbates. Among the other. ingredients added may be natural fatty oils such as olive, coconut, corn, peach, and sesame; natural fats such as stearate and diglycol laurate; hydrocarbon solids, such as paraffin; alcohols such as cetyl, stearyl, and oleyl alcohol; emulsifiers, preservatives, and antioxidants, including vitamin E and paraben; actibacterials, antibacterials and perfumes, especially menthol and camphor.

In one embodiment, the lotion is a protective cream. Protective creams are water-repellent or oil-repellent creams designed to act as barrier ingredients against irritating chemicals, including aqueous chemicals such as, aqueous acids and bases. Some products such as silicones, are both water- and oil-repellent. Among the chemicals used in protective creams are stearic acid, beeswax, glycerin, casein, ammonium hydroxide, zinc stearate, titanium dioxide, butyl stearate, petrolatum, polyethylene glycol, paraffin, potassium hydroxide, magnesium stearate, aluminum compounds, benzoic acid, borates, calamine, ceresin, lanolin, salicylates, sodium silicate, talc, and triethanolamine.

In one embodiment, the lotion is a cleansing cream or liquefying cream. Cleansing creams and lotions are used to dissolve sebum, loosen particles of grime, and to facilitate the removal of dirt. Such creams usually contain mineral oil, triethanolamine stearate, and water. Among other ingredients commonly used are alcohol, alkanoamines, allantoins, antibacterials, and preservatives, methyl and propyl parabens, fatty alcohols, lanolin, perfumes, glycerol, propylene glycol, fatty oils, thickeners, and waxes. One suitable hypoallergenic cold cream contains water, mineral oil, waxes, borax, and depollenixed beeswax. Liquefying Creams are cleansing cream designed to liquefy when rubbed into the skin. Such creams usually contain paraffin, a wax, stearic acid, sodium borate, about 50 to 60 percent liquid petrolatum, and about 20 to 20 percent of water.

In one embodiment the lotion is a foundation or concealer. Cosmetic manufacturers produce a wide assortment of foundations, including oil-free and matte foundations, water-based foundations, oil-based foundations, pressed powder-based foundations, cream-to-powder foundations, liquid-to-powder foundations, stick foundations, so-called self-adjusting foundations, and foundations that have shine, as is disclosed at page 414 of the aforementioned Beauty Book.

As is disclosed at page 250 of the aforementioned “CDCl” of R. Winter, foundation is “aimed at covering blemishes, protecting the skin from drying out, and giving a glowing, healthy look. There is a cream-type foundation that vanishes from the skin but leaves a smooth, protective base for the application of pigmented makeup. Such creams are usually about 75 percent water, 15 percent stearic acid, and the remainder either sorbitan stearate or sorbitol. The pigmented foundation creams which are designed to tint and cover the skin, usually contain about 50 percent water, mineral oil, stearic acid, lanolin, cetyl alcohol, propylene glycol, triethanolamine, borax, and insoluble pigments. They may also contain emulsifiers and detergents; humectants such as propylene glycol, glycerin, and sorbitol to absorb and retain water; lanolin derivatives; perfume; preservatives such as paraben; special barrier ingredients such as zinc stearate; cellulose derivatives and silicone; synthetic esters; thickeners such as sodium alginate, gum tragacanth, quince seed, and mucilage; and such waxes as beeswax and spermaceti. Stick type makeup is made from isopropyl myristate, beeswax, carnauba wax, mineral oil, perfume, and dry pigment. Cake makeup, which is used by applying a wet sponge to the material and then applying the sponge to the face, is usually made of finely ground pigment, talc, kaolin, zinc, titanium oxide, precipitated calcium carbonate, and such inorganic pigments as iron oxides. To these may be added sorbitol, propylene glycol, lanolin, mineral oil, and perfume.”

The “barrier ingredients” referred to therein at page 95 are_“A protective for hand creams and lotions, which acts as a barrier against irritating chemicals, including water and detergents. The water-repellent types deposit a film that acts as a barrier to water and water-soluble ingredients that irritate the skin; oil-repellent types act as barriers against oil and oil-soluble irritants. Silicones are widely used as barrier ingredients. Other skin-protective ingredients in barrier ingredients include petrolatum, paraffin, ozokerite vegetables, beeswax, casein, various celluloses, alginic acid, zein, gum tragacanth, pectin, quince seed, bentonite, zinc oxide, zinc stearate, sodium silicate, talc, stearic acid, and titanium dioxide.”

It is considered common wisdom in cosmetic arts that the wearer's skin and foundation should “match exactly.” Thus, it is often desirable to provide a smoothing appearance with a shear foundation that lets the natural skin color shine though, but still conceals skin blemishes such as wrinkles and scars. One method of accomplishing this is to add a “shine” cosmetic formulation, i.e. one that makes the skin appear to glow. In one embodiment, coating or filling halloysite tubules with a light reflecting material such as mica or silica particles or metals like silver, copper, or aluminum, is done to provide a “shine” formulation. accomplish this.

In some circumstances, facial shine may be perceived by others as oiliness of the wearer's skin. Hence, excessive overall facial shine is undesirable. In one embodiment, the shiny halloysite tubules contain a foundation formulation that is of higher slippage, containing more glycerin and/or silicones than the surrounding foundation or concealer makeup. As is disclosed at pp. 501-502 in the aforementioned “Beauty Bible” of S. Stacy, “Most foundations contain ingredients that allow some amount of movement . . . that also means that those foundations can easily slip into pores . . . Moisturizing when you don't need it creates even more slippage . . . Too much moisturizer or too much foundation can absolutely cause slippage into lines and pores.” This encourages the shiny tubules to aggregate in and around fine lines and wrinkles in the face right where shine is desirable to minimize the appearance of such wrinkles without affecting the overall shine of the face.

To increase the slippage into pores, the carrier lotion may comprise of ingredients traditionally found in foundations but with increased amounts of slippage agents such as glycerin, silicone, and mineral oil.

An appropriate foundation formulation follows, though it may be desired to remove the pigments as well as the waxes which may serve to slow the migration of the carrier lotion to pores. As is disclosed at page 250 of the aforementioned “CDCl” of R. Winter, “The pigmented foundation creams which are designed to tint and cover the skin, usually contain about 50 percent water, mineral oil, stearic acid, lanolin, cetyl alcohol, propylene glycol, triethanolamine, borax, and insoluble pigments. They may also contain emulsifiers and detergents; humectants such as propylene glycol, glycerin, and sorbitol to absorb and retain water; lanolin derivatives; perfume; preservatives such as paraben; special barrier ingredients such as zinc stearate; cellulose derivatives and silicone; synthetic esters; thickeners such as sodium alginate, gum tragacanth, quince seed, and mucilage; and such waxes as beeswax and spermaceti.”

In one embodiment, a suitable carrier is not a lotion but rather a face pack or clay-based mask. In these instances, filled or unfilled halloysite tubes are used to add functionality to face packs and masks. Claims are made for such face masks, and for the thicker face packs, that they shrink pores, remove wrinkles, and relieve tension for the user. Clay face masks usually contain purified siliceous earth, kaolin, glycerin, and water. Face packs usually contain zinc stearate, zinc oxide, tragacanth, alcohol, glycerin, and limewater. Both masks and packs may also contain acacia, Balsam Peru, glyceryl monostearate, magnesium carbonate, wax, salicyclic acid, spermaceti, Turkey-red oil, talc, titanium oxide, and/or zinc sulfate. In various embodiments, the halloysite tubules are filled with one or more of these agents and made a part of the pack or mask formulation. The manner of manufacturing such pack and mask materials is straightforward, as such materials are known to include similar materials such as siliceous earth and kaolin.

In a further embodiment, halloysite tubules are used as mild abrasives in tooth-whitening or skin exfoliation products, such as facial scrubs and clay packs. The tubes in addition to the mild abrasion action may be filled with various active ingredients.

As is disclosed at page 193 of the aforementioned “CDCl” of R. Winter, in the case of dentrifices, “their primary purpose is to clean accessible surfaces of the teeth with a toothbrush. Such cleansing is important to the appearance of the teeth and to gum health, and it prevents mouth odor. Dentifrices usually come in the form of a paste or powder. Despite the brand claims, most dentifrices contain similar ingredients: binders, abrasives, sudsers, humectants, flavors, unique additives, and liquids. Binders include karaya gum, bentonite, sodium alginate, methylcellulose, carrageenan, and magnesium aluminum silicate. Among the abrasives are calcium carbonate, dibasic calcium phosphate, and sodium metaphosphate hydrated alumina. Sudsers include hard soap, and the detergents, sodium lauryl sulfate, sodium lauryl sulfoacetate, dioctyl sodium sulfosuccinate, sulfolaurate, and sodium lauroyl sarcosinate. Humectants include glycerin, propylene glycol, and sorbitol. The most popular flavors are spearmint, peppermint, wintergreen, and cinnamon, but there are also such odd ones as bourbon, rye, anise, clove, caraway, coriander, eucalyptus, nutmeg, and thyme. Fluorides are added to reduce decay; also added are anti-enzyme ingredients (sodium dehydroacetate) and tooth whiteners (sodium perborate). Still other ingredients in dentifrices are sodium benzoate, ammonium antiseptics, sodium coconut monoglyceride sulfonate, sodium copper chlorophyllin, chloroform, starch, sodium chloride, calcium sulfate, strontium chloride, P hydroxybenzoate as a preservative, and sodium dihydroacetate . . . . The most popular toothpaste contains sodium fluoride, calcium pyrophosphate, glycerin, sorbitol, and a blend of anionic surfactants; its competitor contains sodium n-lauroyl sarcosinate and sodium monofluorophosphate . . . as the population is growing older, manufacturers are promoting toothpaste for sensitive teeth and bicarbonate of soda and other ingredients for healthy gums as well as tartar removal.”

Binding ingredients may include the following: Karaya Gum or Sterculia Gum which “is a dried exudate of a tree native to India. Karaya came into wide use during World War I as a cheaper substitute for gum tragacanth. Karaya swells in water and alcohol but does not dissolve. Because of its high viscosity at low concentrations, its ability to produce highly stable emulsions, and its resistance to acids, it is widely used.” (“CDCl” page 269.) Bentonite is “a white clay found in the Midwestern United States and in Canada. Used to thicken lotions, to suspend pigments, and to emulsify oils, and used in makeup lotions, liquid makeup, and facial masks to absorb oil on the face and reduce shine. May clog pores. Also used as a coloring.” (“CDCl” page 101.) Sodium alginate “An emollient . . . It is the sodium salt of alginic acid extracted from brown seaweed. Used in cosmetics as a stabilizer, thickener, and emulsifier.” (“CDCl” page 463.) Alginic acid “is obtained as a highly gelatinous precipitate. The sodium carbonate extracts of brown dried seaweeds are treated with acid to achieve a gelatin. Resembles albumen or gelatin. Alginic acid is slowly soluble in water, forming a very thick liquid” (“CDCl” page 56.) Methylcellulose_or Cellulose Methyl Ether, Citrucel, or Cologel is used as “a binder, thickener, and emulsifying ingredient . . . Introduced in 1947, this compound is prepared from wood pulp or chemical cotton by treatment with alcohol. It swells in water and increases in bulk.” (“CDCl” page 351.) Carrageenan, genus Chondrus crispus or Irish Moss is “a stabilizer and emulsifier, seaweed-like in odor, derived from Irish Moss, used in oils in cosmetics and foods. It is completely soluble in hot water and not coagulated by acids. Used medicinally to soothe the skin. Its use in the United States began in 1935 but really became common during World War II as a replacement for agar-agar.” (“CDCl” page 136.)

Magnesium aluminum silicate, also known as “Fullers Earth,” is another possible binding ingredient. Fuller's Earth is a silver-white, light malleable metal that occurs abundantly in nature and is widely used in combination with various chemicals as a powder.

Additional abrasives include but are not limited to the following:

Calcium carbonate, commonly known as “Chalk. {An] Absorbent that removes shine from talc. A tasteless, odorless powder that occurs naturally in limestone, marble and coral. Used as a white coloring in cosmetics and food, an alkali to reduce acidity, a neutralizer and a firming ingredient, and a carrier for bleaches. Also used in dentifrices as a tooth polisher, in deodorants as a filler, in depilatories as a filler, and in face powder as a buffer. A gastric antacid and anti-diarrhea medicine, it may cause constipation.” (“CDCl” page 126.)

Calcium phosphate, a “White, odorless powder used as an anti-caking ingredient in cosmetics and foods. Employed in toothpaste and tooth powder as an abrasive. Practically insoluble in water.” (“CDCl” page 128.)

Calcium Pyrophosphate is “a fine, white, odorless, tasteless powder used as a nutrient, an abrasive in dentifrices, a buffer, and as a neutralizing ingredient in foodstuffs.” (“CDCl” page 128.).

Sodium Metaphosphate, commonly referred to as Graham's Salt, is “used in dental polishing ingredients, detergents, water softeners, sequestrants, emulsifiers, food additives, and textile laundering. See Sodium Hexylmetaphosphate” (“CDCl” page 472.) Also called Graham's Salt is Sodium Hexylmetaphosphate “used in foods and potable water to prevent scale formation and corrosion. Because it keeps calcium, magnesium, and iron salts solution, it is an excellent water softener and detergent.” (“CDCl” page 469.)

Sudser ingredients, which are generally detergents, include the following:

Soap, which is “usually a mixture of sodium salts of various fatty acids. In liquid soaps, potassium instead of sodium salts is used. Bar soaps vary in contents from brand to brand, depending on the fats or oils used. Sodium hydroxide makes a strong soap, fatty acids a mild soap. So-called neutral soaps are actually alkaline, with pH around 10 (compared to skin which is 5 to 6.5 pH) when dissolved in water . . . . Soap is usually made by the saponification of a vegetable oil with caustic soda. Hard soap consists largely of sodium oleate or sodium palmitate.” (“CDCl” page 462.).

Sodium Lauryl Sulfate is “a detergent, wetting ingredient, and emulsifier . . . Prepared by sulfation of lauryl alcohol followed by neutralization with sodium carbonate. It emulsifies fats.” (“CDCl” page 472.) Dioctyl Sodium Sulfosuccinate or Docustae Sodium is “a waxlike solid that is very soluble in water. It is used as a dispersing and solubilizing ingredient in foods and cosmetics.” (“CDCl” page 209.)

Humectant ingredients, or those that retain moisture in the formulation include the following: Sorbitol which “gives a velvety feel to skin. Used as a replacement for glycerin in emulsions, ointments, . . . and various cosmetic creams. A binder for toilet preparations and a plasticizer and a flavoring ingredient . . . Consists of a white hygroscopic powder flakes or granules with a sweet taste. It is a texturizing ingredient and a sequestrant. Also used in antifreeze, in foods as a sugar substitute, in writing inks to ensure a smooth flow to the point of the pen, and to increase the absorption of vitamins in pharmaceutical preparations.” (“CDCl” page 484.)

Halloysite tubules in dentrifices may also contain active ingredients wherein those ingredients benefit from slow release, timed release, special separation from other ingredients, or protection from degradation during formulation or while on the shelf. Such active ingredients may include flavorings, oral health ingredients, sensitivity ingredients, or bleaching ingredients.

In one embodiment, halloysite tubules contain flavoring ingredients including but not limited to spearmint, wintergreen, peppermint, cinnamon, and menthol. As an example, Spearmint Oil or Mentha viridis “is the essential volatile oil obtained by steam distillation from the fresh above-ground parts of the flowering plant in the United States, Europe, and Asia. It is colorless, yellow or yellow-green, with the characteristic taste and odor of spearmint.” (“CDCl” page 487.)

In one embodiment, halloysite tubules contain oral health ingredients including but not limited to fluorine-containing compounds such as fluoride, sodium fluoride, sodium monofluorophosphate, and/or stannous fluoride; as well as stannous phosphate, zinc picolinate, sodium dehydroacetate, and sarcosine.

Fluoride is “an acid salt used in toothpaste to prevent tooth decay and in nail products to strengthen nails” (“CDCl” page 248.) Sodium Fluoride is “used in toothpastes to prevent tooth decay and as an insecticide, disinfectant, and preservative in cosmetics. Tooth enamel mottling has also been reported.” (“CDCl” page 468.) Sodium Monofluorophosphate is “used in toothpaste to prevent cavities in the U.S.” (“CDCl” page 473.) stannous fluoride also called tin difluoride and Fluoristan, is “prepared by dissolving tin in hydrofluoric acid. Used in dentifrices as a decay preventative.” (“CDCl” page 489.)

Stannous phosphate is a “Salt of tin. It is poorly absorbed from the gastrointestinal tract. Used as an oral care ingredient.” (“CDCl” page 489.) Zinc picolinate is used as an “oral care ingredient” (“CDCl” page 553.) Sodium dehydroacetate or dehydroacetic acid is “a preservative; white, odorless, powdered, with an acrid taste. Used as a plasticizer, fungicide, and bacteria killer in cosmetics; also as an antienzyme ingredient in dentifrices, allegedly to prevent decay.” (“CDCl” page 467.) Sarcosine is “found in starfish and sea urchins, and also formed from caffeine. Sweetish, crystalline acids used in dentifrices as an antienzyme to prevent tooth decay. Because of excellent foaming qualities, they are also used in shampoos.” (“CDCl” page 450.)

In one embodiment, halloysite tubules contain sensitivity ingredients meant to decrease the sensitivity of sensitive teeth. Desensitizing ingredients include but not limited to sodium iodate which is “used in dusting powder and to soothe the skin. White crystalline powder. Antiseptic, particularly to the mucous membranes.” (“CDCl” page 470.)

In one embodiment, halloysite tubules contain bleaching ingredients which are commonly sensitive to degradation by environmental conditions (such as light and air) as well as other ingredients. These ingredients are also known to cause chemical burns and irritation, and thus it is desirable to limit the dosage required for effectiveness, and to minimize damage to surrounding tissues. The bleach filled tubules may also be contained in dental bleaches outside of their use in toothpaste. Dental bleaches_“are products that are put on by your dentists or by toothpaste or products you apply yourself. Usually containing a strong peroxide compound. Eight out of ten dentists now offer bleaching as an esthetic treatment for their patients . . . . In all cases, bleaching benefits vary. The only common adverse effect reported if bleaching is done carefully, is apparently temporary tooth sensitivity. Mild thermal sensitivity (sensitivity to cold) is a common side effect.” (“CDCl” page 192,)

Bleaching ingredients include but are not limited to peroxides, such as e.g. hydrogen peroxide. Peroxides are strong oxidants. Hydrogen peroxide or Peroxyl is “a bleaching and oxidizing ingredient, detergent, and antiseptic . . . . An unstable compound readily broken down into water and oxygen. It is made from barium peroxide and diluted phosphoric acid. Generally recognized as safe as a preservative and germ killer in cosmetics as well as in milk and cheese. A 3 percent solution is used medicinally as an antiseptic and germicide. A strong oxidizer, undiluted it can cause burns of the skin and mucous membranes . . . It is also used in mouthwash gargles.” (“CDCl” page 288.) Sodium perborate consists of “white crystals soluble in water, used as a reingredient, antiseptic, deodorant, bleach, and in dentifrices as a tooth whitener . . . Ulcerations of the mouth have been reported in its use in dentifrices.” (“CDCl” page 475.)

In one embodiment, the halloysite tubules filled or unfilled act as an abrasive in body scrubs and facial peels where the abrasive is used to exfoliate the top layer of dead skin. Exfoliation is the shedding of superficial cells of the skin. Many compounds containing acids are being used in products aimed at the geriatric market as a means of peeling the skin to make it look younger. In the youth market, such products are sold to get rid of oily, blackhead-dotted top layers of skin.

A suitable body scrub formulation to which filled or unfilled halloysite tubules may be added is comprised of purified water, sodium laurel sulfate for a gentle cleansing ingredient derived from coconuts, Cocamide MEA (CAS Registry Number 068140-00-1) for a foaming agent, magnesium aluminum silicate as a thickening agent, apricot seed powder as an exfoliant, aloe vera gel for anti-irritant action, acrylates C10-C30 alkyl acrylate crosspolymer as another thickening agent, aminomethylpropanol to balance pH, fragrance such as menthol, camphor, or mint, sodium citrate as a pH balancer, glycerin for a vegetable emollient, methyl and propyl paraben as preservatives, sodium chloride, and citric acid from citrus fruit of pH balance.

It is, therefore, apparent that there has been provided, in accordance with the present invention, compositions for use in the delivery of a first active agent and a 15 second active agent, the first active agent being sequentially deliverable after said second active agent; and methods for preparation of such compositions. There has been further provided compositions for the removal of unwanted hair from skin, in which the active agents thereof are delivered in sequence. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

We claim:

1. A composition for use in the delivery of a first active agent and a second active agent, said first active agent being sequentially deliverable after said second active agent, said composition comprising:
a. hollow mineral microtubules, each of said hollow mineral microtubules having a length, and opposing ends; and including a cylindrical wall, and a hollow lumen; and a central region, an intermediate region, and an outer region along said length; and
b. within said microtubules, said first active agent disposed within said lumen in said central region of said microtubules, and said second active agent disposed within said lumen in said intermediate region of said microtubules.

2. The composition as recited in claim 1, wherein said hollow mineral microtubules are selected from the group consisting of halloysite, cylindrite, boulangerite, and imogolite and mixtures thereof.

3. The composition as recited in claim 2, wherein said hollow mineral microtubules are halloysite microtubules.

4. The composition as recited in claim 1, wherein said first active agent is a depilatory agent.

5. The composition as recited in claim 4, wherein said second active agent is a moisturizing agent.

6. The composition as recited in claim 4, wherein said second active agent is an anesthetic agent.

7. The composition as recited in claim 1, wherein said hollow mineral microtubules are further comprised of an endcap at each of said opposing ends.

8. The composition as recited in claim 1, wherein said hollow mineral microtubules are dispersed in a carrier fluid.

9. A composition for use in the delivery of a first active agent, a second active agent, and a third active agent, said first active agent being sequentially deliverable after said second active agent, and said second active agent being sequentially deliverable after said third active agent, said composition comprising:
a. hollow mineral microtubules, each of said hollow mineral microtubules having a length, and opposing ends; and including a cylindrical wall, and a hollow lumen; and a central region, an intermediate region, and an outer region along said length; and
b. within said microtubules, said first active agent disposed within said lumen in said central region of said microtubules, said second active agent disposed within said lumen in said intermediate region of said microtubules, and said third active agent disposed within said lumen in said outer region of said microtubules.

10. The composition as recited in claim 9, wherein said hollow mineral microtubules are selected from the group consisting of halloysite, cylindrite, boulangerite, and imogolite and mixtures thereof.

11. The composition as recited in claim 10, wherein said hollow mineral microtubules are halloysite microtubules.

12. The composition as recited in claim 11, wherein said first active agent is a depilatory agent.

13. The composition as recited in claim 12, wherein said second active agent is a moisturizing agent.

14. The composition as recited in claim 13, wherein said third active agent is an anesthetic agent.

15. The composition as recited in claim 9, wherein said hollow mineral microtubules are further comprised of an endcap at each of said opposing ends.

16. The composition as recited in claim 9, wherein said hollow mineral microtubules are dispersed in a carrier fluid.

17. A method for preparing a composition for use in the delivery of a first active agent, and a second active agent, said first active agent being sequentially deliverable after said second active agent, said method comprising the steps of:
a. providing hollow mineral microtubules, each of said hollow mineral microtubules having a length, and opposing ends; and including a cylindrical wall, and a hollow lumen; and a central region, an intermediate region, and an outer region along said length;
b. filling said hollow mineral microtubules with said first active agent;
c. eluting said first active agent in said microtubules, thereby removing said first active agent from said outer region and said intermediate region of said microtubules; and
d. filling said intermediate region with said second active agent.

18. The method as recited in claim 17, further comprising the step of forming an endcap at each of said opposing ends of said microtubules.

19. The method as recited in claim 18, wherein said hollow mineral microtubules are selected from the group consisting of halloysite, cylindrite, boulangerite, and imogolite and mixtures thereof.

20. The method as recited in claim 19, wherein said hollow mineral microtubules are halloysite microtubules.

21. The method as recited in claim 17, further comprising the step of dispersing said microtubules in a carrier fluid.

22. A method for preparing a composition for use in the delivery of a first active agent, a second active agent, and a third active agent, said first active agent being sequentially deliverable after said second active agent, and said second active agent being sequentially deliverable after said third active agent, said method comprising the steps of:
a. providing hollow mineral microtubules, each of said hollow mineral microtubules having a length, and opposing ends; and including a cylindrical wall, and a hollow lumen; and a central region, an intermediate region, and an outer region along said length;
b. filling said hollow mineral microtubules with said first active agent;
c. eluting said first active agent in said microtubules, thereby removing said first active agent from said outer region and said intermediate region of said microtubules;
d. filling said intermediate region and said outer region of said microtubules with said second active agent;
e. eluting said second active agent in said microtubules, thereby removing said second active agent from said outer region of said microtubules; and
f. filling said outer region with said third active agent.

23. The method as recited in claim 22, further comprising the step of forming an endcap at each of said opposing ends of said microtubules.

24. The method as recited in claim 22, wherein said hollow mineral microtubules are selected from the group consisting of halloysite, cylindrite, boulangerite, and imogolite and mixtures thereof.

25. The method as recited in claim 24, wherein said hollow mineral microtubules are halloysite microtubules.

26. The method as recited in claim 22, further comprising the step of dispersing said microtubules in a carrier fluid.
